(19) United States
(12) Patent Application Publication
(10) Pub. No.: US 2023/0135434 A1
(43) Pub. Date: May 4, 2023

(54) COMPOSITIONS COMPRISING BACTERIAL STRAINS

(71) Applicant: 4D Pharma Research Limited, Aberdeen (GB)

(72) Inventors: Imke Elisabeth MULDER, Aberdeen (GB); Nicole REICHARDT, Aberdeen (GB); Helene SAVIGNAC, Aberdeen (GB); Sasha CHETAL, Aberdeen (GB); Ted DINAN, Cobh (IE); John CRYAN, Cork (IE)

(21) Appl. No.: 17/748,096

(22) Filed: May 19, 2022

Related U.S. Application Data
(63) Continuation of application No. PCT/EP2020/082991, filed on Nov. 20, 2020.
Application No. PCT/EP2020/082991 is a continuation of application No. PCT/EP2020/063854, filed on May 18, 2020.

(30) Foreign Application Priority Data
Nov. 20, 2019 (EP) 19210481.8
Dec. 20, 2019 (GB) 1919003.2
Dec. 23, 2019 (GB) 1919227.7
Dec. 30, 2019 (GB) 1919420.8
Feb. 28, 2020 (GB) 2002902.1

(51) Int. Cl. A61K 35/74

(52) U.S. Cl. CPC A61K 35/74

(57) ABSTRACT

The invention provides compositions comprising a bacterial strain of the genus Fusicatenibacter and their use in medicine.

### CROSS-REFERENCE

This application is a continuation of International Application No. PCT/EP2020/082991, filed Nov. 20, 2020, which is a continuation of International Application No. PCT/EP2020/063854, filed May 18, 2020, which claims the benefit of European Application No. 19210481.8, filed Nov. 20, 2019, Great Britain Application No. 1919003.2, filed Dec. 20, 2019, Great Britain Application No. 1919227.7, filed Dec. 23, 2019, Great Britain Application No. 1919420.8, filed Dec. 30, 2019, and Great Britain Application No. 2002902.1, filed Feb. 28, 2020, all of which are hereby incorporated by reference in their entirety.

### SEQUENCE LISTING

The instant application contains a Sequence Listing which has been submitted electronically in ASCII format and is hereby incorporated by reference in its entirety. Said ASCII copy, created on May 16, 2022, is named 56708_758_301_SL.txt and is 8,175 bytes in size.

### TECHNICAL FIELD

This invention is in the field of compositions comprising bacterial strains and the use of such compositions in therapy.

### BACKGROUND TO THE INVENTION

The human intestine is thought to be sterile in utero, but it is exposed to a large variety of maternal and environmental microbes immediately after birth. Thereafter, a dynamic period of microbial colonization and succession occurs, which is influenced by factors such as delivery mode, environment, diet and host genotype, all of which impact upon the composition of the gut microbiota, particularly during early life. Subsequently, the microbiota stabilizes and becomes adult-like [1]. The human gut microbiota contains more than 1,500 different phylotypes dominated in abundance levels by two major bacterial divisions (phyla), the Bacteroidetes and the Firmicutes [2-3]. The successful symbiotic relationships arising from bacterial colonization of the human gut have yielded a wide variety of metabolic, structural, protective and other beneficial functions. The enhanced metabolic activities of the colonized gut ensure that otherwise indigestible dietary components are degraded with release of by-products providing an important nutrient source for the host and additional health benefits. Similarly, the immunological importance of the gut microbiota is well-recognized and is exemplified in germ-free animals which have an impaired immune system that is functionally reconstituted following the introduction of commensal bacteria [4-6].

Dramatic changes in microbiota composition have been documented in gastrointestinal disorders such as inflammatory bowel disease (IBD). For example, the levels of Clostridium cluster XIVa and Clostridium cluster XI (F. prausnitzii) bacteria are reduced in IBD patients whilst numbers of E. coli are increased, suggesting a shift in the balance of symbionts and pathobionts within the gut [7-11].

In recognition of the potential positive effect that certain bacterial strains may have on the animal gut, various strains have been proposed for use in the treatment of various diseases (see, for example, [12-15]). A number of strains, including mostly Lactobacillus and Bifidobacterium strains, have been proposed for use in treating various bowel disorders (see [16] for a review). The use of heat killed Fusicatenibacter saccharivorans has been suggested to decrease ulcerative colitis [17].

WO2017/182796 teaches the isolation of various bacterial species from the gut and speculates on the use of these bacteria to treat pathogenic infection such as C. difficile. US 2019/0030098 discusses the use of compositions comprising two or more purified bacterial strains in the treatment of a pathogenic infection such as C. difficile. WO2018/067887 relates to methods of diagnosing fungal dysbiosis in the gut of a subject with cirrhosis that involve assaying a sample from the subject for bacterial taxa. It also speculates that dysbiosis can be treated with probiotics but there is no indication regarding the composition of such probiotics.

Bacteria show great promise in the treatment of a large variety of diseases and there is a requirement for new therapies using bacteria to be developed.

### SUMMARY OF THE INVENTION

The inventors have developed new therapies for treating and preventing a central nervous system disease or disorder, a disease which is associated with increased intestinal permeability, an inflammatory disease, a metabolic disease or a cardiovascular disease. The inventors have demonstrated that compositions comprising a bacterial strain from the genus Fusicatenibacter are particularly effective in the treatment of stereotyped, social behaviour and depressive-like behaviours in murine models of autism.

The invention provides compositions comprising bacteria from the genus Fusicatenibacter for use in treating or preventing a central nervous system disease or disorder, a disease which is associated with increased intestinal permeability, an inflammatory disease, a metabolic disease or a cardiovascular disease. The invention also provides compositions comprising a bacterial strain that has a 16s rRNA sequence that has at least 98.65% sequence similarity to SEQ ID NOs:1, 2, 3 or 4 for use in a method of treating or preventing a central nervous system disease or disorder, a disease which is associated with increased intestinal permeability, an inflammatory disease, a metabolic disease or a cardiovascular disease. The central nervous system disease or disorder can be a neurodegenerative disease, a neurodevelopmental disorder or a neuropsychiatric condition.

Additionally, the invention provides methods of treating or preventing a central nervous system disease or disorder, a disease which is associated with increased intestinal permeability, an inflammatory disease, a metabolic disease or a cardiovascular disease, comprising administering a composition comprising a bacterial strain of the genus Fusicatenibacter.

Furthermore, the invention provides uses of a composition comprising a bacterial strain of the genus Fusicatenibacter for the manufacture of a medicament for the treatment or prevention a central nervous system disease or disorder, a disease which is associated with increased intestinal permeability, an inflammatory disease, a metabolic disease or a cardiovascular disease.

The central nervous system disease or disorder may be mediated by the microbiota-gut-brain axis. In certain embodiments, the composition is for use in a method of modulating the microbiota-gut-brain axis.

The invention also provides compositions, uses and methods comprising a bacterial strain of the genus Fusicatenibacter, for use in a method of treating or preventing a neurodevelopmental disorder or a neuropsychiatric condition. The neurodevelopmental disorder or neuropsychiatric condition may be selected from the group consisting of autism spectrum disorders (ASDs); child developmental disorder; obsessive compulsive disorder (OCD); major depressive disorder; depression; seasonal affective disorder; anxiety disorders; chronic fatigue syndrome (myalgic encephalomyelitis); stress disorder; post-traumatic stress disorder; schizophrenia spectrum disorders; schizophrenia; bipolar disorder; psychosis; mood disorder; chronic pain; Guillain-Barré syndrome and meningitis, dementia, including Lewy body, vascular and frontotemporal dementia; primary progressive aphasia; mild cognitive impairment; HIV-related cognitive impairment, and corticobasal degeneration.

In preferred embodiments, the invention provides compositions for use in treating or preventing autism.

The composition of the invention is preferably an oral composition. The inventors have shown that oral administration of a composition of the invention can be effective for treating the conditions disclosed herein, in particular inflammatory central nervous system diseases and disorders, in particular those mediated by the microbiota-gut-brain axis. Also, oral administration is convenient for patients and practitioners and allows delivery to and/or partial or total colonisation of the intestine.

The composition of the invention may comprise one or more pharmaceutically acceptable excipients or carriers. In certain embodiments, the composition of the invention has been lyophilised. The composition of the invention can also comprise a lyophilised bacteria strain of the genus Fusicatenibacter. The bacterial strain may have been lyophilised. Lyophilisation is an effective and convenient technique for preparing stable compositions that allow delivery of bacteria. In particularly preferred embodiments, the bacterial strain is viable and capable of partially or totally colonising the intestine.

The invention further provides a food product comprising a composition comprising a bacterial strain of the genus Fusicatenibacter as described above. The invention also provides a vaccine composition comprising a bacterial strain of the genus Fusicatenibacter as described above.

The inventors have identified and characterised a Fusicatenibacter saccharivorans bacterial strain that is particularly useful for therapy. The Fusicatenibacter saccharivorans species of the invention are shown to be effective for treating the diseases described herein, such as central nervous system diseases or disorders. Therefore, in another aspect, the invention provides a cell of the Fusicatenibacter saccharivorans strain deposited under accession number NCIMB 43527, or a derivative thereof. The invention also provides compositions comprising such cells, or biologically pure cultures of such cells. The invention also provides a cell of the Fusicatenibacter saccharivorans strain deposited under accession number NCIMB 43527, or a derivative thereof, for use in therapy, in particular for the diseases described herein.

### BRIEF DESCRIPTION OF DRAWINGS

FIG. 1 shows the permeability of ileum (Top) and colon (Bottom), measured using fluorescence.

FIGS. 2A-2C shows the expression of IDO1 (FIG. 2A) and TPH1 (FIG. 2B) in the ileum, and of TPH1 in the colon (FIG. 2C), normalized to β-actin. *=p≤0.05

FIGS. 3A-3F shows the concentration of the short-chain fatty acids acetate (FIG. 3A), propionate (FIG. 3B), isobutyrate (FIG. 3C), butyrate (FIG. 3D), isovalerate (FIG. 3E) and valerate (FIG. 3F) in the caecal content.

FIGS. 4A-4G shows expression BDNF (FIG. 4A), Grin2a (FIG. 4B), glucocorticoid receptor (FIG. 4C), CRFR2 (FIG. 4D) and IL-6 (FIG. 4E) in the amygdala and of TLR-4 (FIG. 4F) and BDNF (FIG. 4G) in the PFC, normalised to β-actin, in the amygdala. *=p<0.05.

FIGS. 5A-5B shows marble burying behaviour in the Btrb (FIG. 5A) and MIA (FIG. 5B) mice, expressed as the number of marbles buried. *=p<0.05.

FIGS. 6A-6F shows the set up of the experiment to measure time spent with social stimuli vs non-social stimuli (FIG. 6A) and time spent exploring a novel animal vs familiar animal (FIG. 6B). The duration is expressed in s. Panels in FIG. 6C and FIG. 6D show the results for Btbr mice and panels in FIG. 6E and FIG. 6F show the results for MIA mice. *=p<0.05; **=p<0.01; ***=p<0.001 and ****=p<0.0001.

FIGS. 7A-7B shows the results of the forced swim test for Btbr (FIG. 7A) and MIA (FIG. 7B) mice. **=p<0.01 and ****=p<0.0001.

FIGS. 8A-8B shows the results of the intestinal mobility test in Btbr (FIG. 8A) and MIA (FIG. 8B) mice.

FIG. 9 shows the permeability of ileum and colon in Btbr and MIA mice after treatment with Fusicatenibacter saccharivorans.

### DISCLOSURE OF THE INVENTION

Bacterial Strains

The compositions of the invention comprise a bacterial strain of the genus Fusicatenibacter. In embodiments, they comprise a live bacterial strain of the genus Fusicatenibacter. Preferably, the bacterial strain in the composition of the invention is viable. Preferably the bacterial strain capable of partially or totally colonising the intestine. Preferably, the bacterial strain in the composition of the invention is viable and capable of partially or totally colonising the intestine. The examples demonstrate that bacteria of this genus (in particular live bacteria) are useful in methods for treating or preventing diseases such as a central nervous system disease or disorder, a disease which is associated with increased intestinal permeability, an inflammatory disease, a metabolic disease or a cardiovascular disease. In preferred embodiments, the bacteria from the genus Fusicatenibacter are for use in treating or preventing autism.

Examples of Fusicatenibacter species for use in the invention include Fusicatenibacter saccharivorans. Fusicatenibacter species are gram-reaction-positive, obligate anaerobes that are non-motile, non-spore-forming and spindle-shaped. Fusicatenibacter species can produce lactic acid, formic acid, acetic acid and succinic acid as fermentation end products from glucose. As demonstrated in the examples, bacteria from the genus Fusicatenibacter have newly been identified as butyrate producers.

Fusicatenibacter may be isolated from the human gut. Examples of Fusicatenibacter saccharivorans strains isolated from human faeces include HT03-11T, KO-38 and TT-111 [18]. The type strain of Fusicatenibacter saccharivorans is HT03-11^{T} (=YIT 12554^{T}=JCM 18507^{T}=DSM 26062^{T}). The GenBank accession number for the 16S rRNA gene sequence of Fusicatenibacter saccharivorans strains HT03-11T, KO-38 and TT-111 are AB698910, AB698914 and AB698915, respectively (disclosed herein as SEQ ID NO: 1-3).

A preferred Fusicatenibacter saccharivorans strain is the strain deposited under accession number NCIMB 43527, which is also referred to herein as Ref 1.

A 16S rRNA sequence for the Ref 1 strain is provided in SEQ ID NO:4. The strain was deposited with the international depositary authority NCIMB, Ltd. (Ferguson Building, Craibstone Estate, Bucksburn, Aberdeen, AB21 9YA, Scotland) by 4D Pharma Research Limited (Life Sciences Innovation Building, Aberdeen, AB25 2ZS, Scotland) on 3 Dec. 2019 and was assigned accession number NCIMB 43527.

In developing the invention, the inventors have identified and characterised a bacterial genus that is particularly useful for therapy. The data disclosed herein suggest that the genus Fusicatenibacter may be useful for therapy, in particular treating or preventing the diseases described herein, such as a disease or disorder selected from the group consisting of: a central nervous system disease, disorder or condition, impaired gut barrier function, a disease which is associated with increased intestinal permeability, an inflammatory disease, a metabolic disease and a cardiovascular disease. Therefore, in another aspect, the invention provides a cell of a Fusicatenibacter strain deposited under accession numbers NCIMB 43527, or a derivative thereof, e.g. for a use or method as disclosed herein. The invention also provides a composition comprising such cells, or biologically pure cultures of such cells, e.g. for a use or method as disclosed herein.

In preferred embodiments, the composition comprises a strain deposited under accession numbers NCIMB 43527, for use in therapy, such as a method of treating or preventing a disease or disorder selected from the group consisting of: a central nervous system disease, disorder or condition, impaired gut barrier function, a disease which is associated with increased intestinal permeability, an inflammatory disease, a metabolic disease and a cardiovascular disease. Exemplary uses include: treating or preventing a neurodevelopmental disorder or a neuropsychiatric condition; and/or treating or preventing autism spectrum disorder, preferably autism. In especially preferred embodiments, the composition comprising a strain deposited under accession number NCIMB 43527 may be used in a method of preventing, reducing or alleviating one or more stereotyped, repetitive, compulsive and/or anxious behaviour, especially in the treatment of autism.

Bacterial strains closely related to the strain tested in the examples are also expected to be effective for use in treating and preventing the diseases mentioned herein, and in particular central nervous system diseases or disorders. In certain embodiments, the bacterial strain for use in the invention has a 16s rRNA sequence that is at least 95%, 96%, 97%, 98%, 99%, 99.5%, 99.6%, 99.7%, 99.8% or 99.9% identical to the 16s rRNA sequence of a bacterial strain of Fusicatenibacter saccharivorans. Preferably, the bacterial strain for use in the invention has a 16s rRNA sequence that is at least 95%, 96%, 97%, 98%, 99%, 99.5%, 99.6%, 99.7%, 99.8% or 99.9% identical to any one of SEQ ID NOs:1-4. Preferably, the bacterial strain for use in the invention has a 16s rRNA sequence that is at least 95%, 96%, 97%, 98%, 99%, 99.5%, 99.6%, 99.7%, 99.8% or 99.9% identical to SEQ ID NOs: 1, 2, 3 or 4.

Preferably, the bacterial strain has a 16s rRNA sequence that has at least 98.65% sequence similarity to SEQ ID NOs: 1, 2, 3 or 4. Pairwise similarities between 16S rRNA gene sequences can be calculated based on robust global sequence alignment algorithms such as the EzTaxon server described in [19].

Bacterial strains that are biotypes of the HT03-11T, KO-38, TT-111 or NCIMB 43527 strains mentioned above are also expected to be effective for use in treating and preventing the diseases mentioned herein, and in particular central nervous system diseases or disorders. A biotype is a closely related strain that has the same or very similar physiological and biochemical characteristics, as defined herein.

Strains that are biotypes of the HT03-11T, KO-38, TT-1 11 or NCIMB 43527 strains or the bacterial strain deposited under accession number NCIMB 43527 and that are suitable for use in the invention may be identified by sequencing other nucleotide sequences for those strains. For example, substantially the whole genome may be sequenced and a biotype strain for use in the invention may have at least 95%, 96%, 97%, 98%, 99%, 99.5%, 99.6%, 99.7%, 99.8% or 99.9% sequence identity across at least 80% of its whole genome (e.g. across at least 85%, 90%, 95% or 99%, or across its whole genome). For example, in some embodiments, a biotype strain has at least 98% sequence identity across at least 98% of its genome or at least 99% sequence identity across 99% of its genome. Other suitable sequences for use in identifying biotype strains may include hsp60 or repetitive sequences such as BOX, ERIC, (GTG)₅ or REP [20]. Biotype strains may have sequences with at least 95%, 96%, 97%, 98%, 99%, 99.5%, 99.6%, 99.7%, 99.8% or 99.9% sequence identity to the corresponding sequence of the HT03-11T, KO-38, TT-111 or NCIMB 43527 strains. In some embodiments, a biotype strain has a sequence with at least 97%, 98%, 99%, 99.5%, 99.6%, 99.7%, 99.8% or 99.9% sequence identity to the 16S rRNA sequence of SEQ ID NOs: 1, 2, 3 or 4.

In certain embodiments, the bacterial strain for use in the invention has a genome with at least 90% sequence identity (e.g. at least 92%, 94%, 95%, 96%, 97%, 98%, 99% or 100% sequence identity) to the genome of Fusicatenibacter saccharivorans across at least 60% (e.g. at least 65%, 70%, 75%, 80%, 85%, 95%, 96%, 97%, 98%, 99% or 100%) of the genome.

Alternatively, strains that are biotypes of the HT03-11T, KO-38, TT-111 or NCIMB 43527 strains and that are suitable for use in the invention may be identified by using restriction fragment analysis and/or PCR analysis, for example by using fluorescent amplified fragment length polymorphism (FAFLP) and repetitive DNA element (rep)-PCR fingerprinting, or protein profiling, or partial 16S or 23s rDNA sequencing. In preferred embodiments, such techniques may be used to identify other Fusicatenibacter saccharivorans strains.

In certain embodiments, strains that are biotypes of the HT03-11T, KO-38, TT-111 or NCIMB 43527 strains that are suitable for use in the invention are strains that provide the same pattern as the HT03-1IT, KO-38, TT-1 11 or NCIMB 43527 strains respectively when analysed by amplified ribosomal DNA restriction analysis (ARDRA), for example when using Sau3AI restriction enzyme (for exemplary methods and guidance see, for example [21]). Alternatively, biotype strains are identified as strains that have the same carbohydrate fermentation patterns as the HT03-11T, KO-38, TT-1 11 or NCIMB 43527 strains.

Other Fusicatenibacter strains or Fusicatenibacter saccharivorans strains in particular that are useful in the invention, such as biotypes of the HT03-11T, KO-38, TT-111 or NCIMB 43527 strains, may be identified using any appropriate method or strategy, including the assays described in the examples. For instance, strains for use in the invention may be identified by culturing bacteria and administering them to MIA or BTBR mice before completing marble burying, social behaviour and/or forced swim tests. In another example, strains for use in the invention may be identified by culturing bacteria and assessing their short-chain fatty acid (SCFA) production profile; biotypes which may be employed in the present invention may produce butyrate, valerate and/or isovalerate. Additionally, strains for use in the invention may be identified by administering them to murine models and observing their effect on intestinal permeability. In particular, bacterial strains that have similar growth patterns, metabolic type and/or surface antigens to the HT03-11T, KO-38, TT-111 or NCIMB 43527 strains may be useful in the invention. A useful strain will have comparable activity to the HT03-11T, KO-38, TT-1 11 or NCIMB 43527 strain as assessed, for example, by marble burying, social behaviour and forced swim tests in mice models. In particular, a biotype strain will elicit comparable effects on gut permeability and/or on SCFA production to the effects shown in the examples, which may be identified by using the culturing and administration protocols described in the examples. For example, a biotype strain will be able to decrease intestinal permeability of Btbr or MIA mice by at least about 10%, at least about 20% or at least about 50%±10% compared to a control when measured using Ussing chambers with oxygenated (95% O2, 5% CO2) Krebs buffer maintained at 37° C. In addition, or alternatively, it may increase SCFA production by at least about 10, at least about 20, or at least about 50% compared to a control when administered to Balb mice when measured using a Varian 3500 GC flame-ionization system, fitted with a with a ZB-FFAP column.

A strain identified using 16S homology and MALDI-TOF analysis as belonging to the Fusicatenibacter saccharivorans species has been tested in the examples and shown to be effective for treating disease. This strain is particularly preferred and has been deposited under accession number NCIMB 43527. Therefore, the invention provides a cell, such as an isolated cell, of the Fusicatenibacter saccharivorans species for use in therapy, in particular for the diseases described herein. The invention also provides a cell, such as an isolated cell, of the Fusicatenibacter saccharivorans species, most preferably the one deposited under accession number NCIMB 43527, or a derivative thereof, for use in therapy, in particular for the diseases described herein.

A derivative of the HT03-11T, KO-38, TT-111 or NCIMB 43527 strains may be a daughter strain (progeny) or a strain cultured (subcloned) from the original. A derivative of a strain of the invention may be modified, for example at the genetic level, without ablating the biological activity. In particular, a derivative strain of the invention is therapeutically active. A derivative strain will have comparable microbiota modulatory activity to the HT03-11T, KO-38, TT-111 or NCIMB 43527 strains. In particular, a derivative strain will elicit comparable effects on autism model to the effects shown in the examples, which may be identified by using the culturing and administration protocols described in the examples.

The bacterial strain may also be a strain that has the same safety and therapeutic efficacy characteristics as the HT03-11T, KO-38, TT-111 or NCIMB 43527 strains.

The invention also provides a cell of the Fusicatenibacter saccharivorans strain deposited under accession number NCIMB 43527, or derivatives thereof, for use in therapy, in particular for the diseases described herein.

Therapeutic Uses

The invention provides compositions for use in a method of treating or preventing a central nervous system disease or disorder, an inflammatory disease, a metabolic disease or a cardiovascular disease. A central nervous system disease or disorder can include a neurodevelopmental disorder, a neurodegenerative disease or a neuropsychiatric condition. In preferred embodiments, the compositions of the invention are for use in a method of treating or preventing autism spectrum disorders.

Preferably, a composition of the invention is not for use in treating or preventing an infectious disease, e.g. a microbial infection such as Clostridium Difficile infection. Preferably, the subject to be treated does not suffer from or is at risk of a Clostridium Difficile infection.

The neurodevelopmental disorder or neuropsychiatric condition can be selected from the group consisting of autism spectrum disorders (ASDs); child developmental disorder; obsessive compulsive disorder (OCD); major depressive disorder; depression; seasonal affective disorder; anxiety disorders; chronic fatigue syndrome (myalgic encephalomyelitis); stress disorder; post-traumatic stress disorder; schizophrenia spectrum disorders; schizophrenia; bipolar disorder; psychosis; mood disorder; chronic pain; Guillain-Barré syndrome and meningitis, dementia, including Lewy body, vascular and frontotemporal dementia; primary progressive aphasia; mild cognitive impairment; HIV-related cognitive impairment; and corticobasal degeneration.

The neurodegenerative disease can be selected from the group consisting of Parkinson's disease, including progressive supranuclear palsy, progressive supranuclear palsy, Steele-Richardson-Olszewski syndrome, normal pressure hydrocephalus, vascular or arteriosclerotic parkinsonism and drug-induced parkinsonism; Alzheimer's disease, including Benson's syndrome; multiple sclerosis; Huntington's disease; amyotrophic lateral sclerosis; Lou Gehrig's disease; motor inflammatory neurone disease; prion disease; spinocerebellar ataxia and spinal muscular atrophy.

The compositions of the invention are for use in a method of treating or preventing diseases associated with intestinal permeability, such as gastric ulcers, infectious diarrhea, irritable bowel syndrome, functional GI diseases, inflammatory bowel disease, celiac disease and cancer (esophagus, colorectal), food allergies, acute inflammation (sepsis, systemic inflammatory response syndrome (SIRS), multiple organ failure), obesity, metabolic diseases (e.g. NAFLD and NASH).

The compositions of the invention are for use in a method of treating or preventing metabolic diseases, such as obesity and diabetes. The compositions of the invention are for use in a method of treating or cardiovascular diseases, such as atherosclerosis, hypertension, coronary artery disease, angina and myocardial infarction, heart arrhythmia, heart failure, valvular heart disease, pericardial disease, stroke, hypertensive heart disease, rheumatic heart disease, cardiomyopathy, congenital heart disease, carditis, aortic aneurysms, peripheral artery disease, thromboembolic disease, and venous thrombosis.

The compositions of the invention may be useful for treating or preventing a central nervous system disease or disorder, an inflammatory disease, a metabolic disease or a cardiovascular disease in adult patients. In preferred embodiments, the patients may be an adult aged between 18 and 65 years old, for example between 40 to 65 years old or older than 40 years. The adult patient may 18 years, 20 years, 30 years, 40 years, 50 years, 60 years, 70 years or older. The adult patient may be aged between 18 to 70 years old, 18 to 50 years old or 18 to 30 years old. The compositions of the invention may be for use in treating a disease associated with old age, for example, a disease diagnosed in a patient who is older than 50 years. The compositions of the invention may be effective at treating central nervous system disease or disorder, such as neurodegenerative diseases that occur in elderly patients, for example, the patients are 65 years or older.

The compositions of the invention may be useful for treating or preventing a central nervous system disease or disorder, an inflammatory disease, a metabolic disease or a cardiovascular disease in children, for example in adolescents and infants. The patients may be a child between 1 and 17 years old, for example between 1 to 2 years old, 2 to 5 years old, 5 to 7 years old, 5 to 9 years old, 7 to 11 years old, older than 5 years, older than 7 years old, old than 10 years old, older than 15 years old. Early diagnosis and intervention of autism spectrum disorders is recommended [22], therefore the patient may be 9 to 24 months old or 18 to 24 months old.

Modulation of the Microbiota-Gut-Brain Axis

Communication between the gut and the brain (the microbiota-gut-brain axis) occurs via a bidirectional neurohumoral communication system. Recent evidence shows that the microbiota that resides in the gut can modulate brain development and produce behavioural phenotypes via the microbiota-gut-brain axis. Indeed, a number of reviews suggest a role of the microbiota-gut-brain axis in maintaining central nervous system functionality and implicate dysfunction of the microbiota-gut-brain axis in the development of central nervous system disorders and conditions [10],[13],[14],[23].

The bidirectional communication between the brain and the gut (i.e. the-gut-brain axis) includes the central nervous system, neuroendocrine and neuroimmune systems, including the hypothalamus-pituitary-adrenal (HPA) axis, sympathetic and parasympathetic arms of the autonomic nervous system (ANS), including the enteric nervous system (ENS) and the vagus nerve, and the gut microbiota.

As demonstrated in the examples, the compositions of the invention can modulate the microbiota-gut-brain axis and reduce behavioural symptoms associated with a CNS disorder. Accordingly, the compositions of the invention may be useful for treating or preventing disorders of the central nervous system (CNS), in particular those disorders and conditions associated with dysfunction of the microbiota-gut-brain axis.

The compositions of the invention are also useful for treating or preventing neurodevelopmental disorders and/or neuropsychiatric conditions. Neurodevelopmental diseases and neuropsychiatric conditions are often associated with the microbiota-gut-brain axis. The compositions of the invention may be useful for treating or preventing neurodevelopmental diseases and/or neuropsychiatric conditions mediated by dysfunction of the microbiota-gut-brain axis. In further preferred embodiments, the compositions of the invention are for use in treating or preventing a neurodevelopmental disorder or a neuropsychiatric condition.

In particular embodiments, the compositions of the invention may be useful for treating or preventing a disease or condition selected from the group consisting of: autism spectrum disorders (ASDs); child developmental disorder; obsessive compulsive disorder (OCD); major depressive disorder; depression; seasonal affective disorder; anxiety disorders; schizophrenia spectrum disorders; schizophrenia; bipolar disorder; psychosis; mood disorder; chronic fatigue syndrome (myalgic encephalomyelitis); stress disorder; post-traumatic stress disorder; dementia; Alzheimer's; Parkinson's disease; and/or chronic pain. In further embodiments, the compositions of the invention may be useful for treating or preventing motor neuron disease; Huntington's disease; Guillain-Barré syndrome and/or meningitis.

The compositions of the invention may be particularly useful for treating or preventing chronic disease, treating or preventing disease in patients that have not responded to other therapies (such as treatment with anti-psychotics and/or anti-depressants), and/or treating or preventing the tissue damage and symptoms associated with dysfunction of the microbiota-gut-brain axis.

In certain embodiments, the compositions of the invention modulate the CNS. In some embodiments, the compositions of the invention modulate the autonomic nervous system (ANS). In some embodiments, the compositions of the invention modulate the enteric nervous system (ENS). In some embodiments, the compositions of the invention modulate the hypothalamic, pituitary, adrenal (HPA) axis. In some embodiments, the compositions of the invention modulate the neuroendocrine pathway. In some embodiments, the compositions of the invention modulate the neuroimmune pathway. In some embodiments, the compositions of the invention modulate the CNS, the ANS, the ENS, the HPA axis and/or the neuroendocrine and neuroimmune pathways.

The signalling of the microbiota-gut-brain axis is modulated by neural systems. Accordingly, in some embodiments, the compositions of the invention modulate signalling in neural systems. In certain embodiments, the compositions of the invention modulate the signalling of the central nervous system. In some embodiments, the compositions of the invention modulate signalling in sensory neurons. In other embodiments, the compositions of the invention modulate signalling in motor neurons. In some embodiments, the compositions of the invention modulate the signalling in the ANS. In some embodiments, the ANS is the parasympathetic nervous system. In preferred embodiments, the compositions of the invention modulate the signalling of the vagus nerve. In other embodiments, the ANS is the sympathetic nervous system. In other embodiments, the compositions of the invention modulate the signalling in the enteric nervous system. In certain embodiments, the signalling of ANS and ENS neurons responds directly to luminal contents of the gastrointestinal tract. In other embodiments, the signalling of ANS and ENS neurons responds indirectly to neurochemicals produced by luminal bacteria. In other embodiments, the signalling of ANS and ENS neurons responds to neurochemicals produced by luminal bacteria or enteroendocrine cells. In certain preferred embodiments, the neurons of the ENS activate vagal afferents that influence the functions of the CNS. In some embodiments, the compositions of the invention regulate the activity of enterochromaffin cells.

In certain embodiments, the compositions of the invention modulate fear conditioning in an animal model. In certain embodiments, the compositions of the invention can be used to modulate the development of fear and/or anxiety, and/or modulate the extent to which the fear and/or anxiety becomes extinct in a subject. In certain embodiments, the compositions of the invention can be used to modulate the extent of stress-induced hyperthermia in an animal model. In certain embodiments, the compositions of the invention modulate the level of stress and/or anxiety in a subject.

The examples demonstrate that the compositions of the invention can increase the activation of BDNF. BDNF acts on certain neurons of the central nervous system to support the survival of existing neurons and help the growth and development of new neurons and synapses. BDNF is active in the hippocampus, cortex and basal forebrain, and is important for long-term memory. The compositions of the invention can therefore be useful to increase the secretion of BDNF. The compositions may therefore be used in the treatment of neurodegenerative diseases and/or neuropsychiatric conditions associated with the impairment of long-term memory. The compositions of the invention may be used for improving long-term memory, in particular for improving long-term memory that is impaired by a neurodegenerative disease and/or neuropsychiatric conditions.

Increased levels of kynurenine have been shown to attenuated MPP+-induced neuronal cell death in vitro in a human dopaminergic neuroblastoma cell line [24]. In certain embodiments kynurenine and kynurenic acid, can activate GI aryl hydrocarbon receptor (Ahr) and GPR35 receptors. Activation of Ahr receptor induces IL-22 production, which can inhibit local inflammation. Activation of GPR35 inducing the production of inositol triphosphate and Ca2+ mobilization.

IDO-deficient mice and mice treated with IDO inhibitors fail to tolerate the foetus during allogeneic pregnancy, experience colitis due to the failure of mucosal tolerance of the intestinal microbiota, and lose the ability to clear apoptotic cells. Furthermore, blocking or ablating IDO expression worsens inflammation in models of graft-versus-host disease, autoimmunity, and chronic conditions, such as chronic granulomatous disease and diabetes, illustrating the key role played by IDO in controlling inflammation [25]. The examples demonstrate that compositions of the invention can increase IDO expression. The compositions of the invention may be useful for treating diseases or disorders, such as a central nervous system disease, or disorder or condition, impaired gut barrier function, a disease which is associated with increased intestinal permeability, an inflammatory disease, a metabolic disease and a cardiovascular disease.

Neurodevelopmental Disorders or Neuropsychiatric Conditions

The invention also provides compositions comprising a bacterial strain of the genus Fusicatenibacter, for use in a method of treating or preventing a neurodevelopmental disorder or a neuropsychiatric condition.

The neurodevelopmental disorder or neuropsychiatric condition can be selected from an autism spectrum disorder, an obsessive compulsive disorder, a major depressive disorder, an anxiety disorder or an inflammatory neurocognitive disorder.

Autism Spectrum Disorder (ASD)

Autism spectrum disorder is a set of heterogeneous neurodevelopmental conditions, characterised by early-onset difficulties in social interaction, communication and unusually restricted, repetitive behaviour and interests. Symptoms can be recognised from a very early age but ASD is often diagnosed in more able children starting mainstream education. Autism represents the primary type of ASD.

Historically, autism has been diagnosed on the basis of three core domains: impaired social interaction, abnormal communication, and restricted and repetitive behaviours and interests. In the International Classification of Diseases (ICD-10R, WHO 1993) and the Diagnostic and Statistical Manual (DSM-IV, American Psychiatric Association, 2000), autism comes under the umbrella term of Pervasive Developmental Disorder (PDD), with four possible diagnostic subtypes: Asperger Syndrome, Childhood Autism/Autistic Disorder, Atyptical Autism, and PDD—not otherwise specified. In DMS-5, these diagnostic subtypes are combined into a single category of autism spectrum disorder (ASD) and the previous use of three core domains of impairment has been reduced to two main areas, namely social communication and interaction, and repetitive behaviour, which include sensory integration dysfunctions.

ASD is a ‘spectrum disorder’ as it affects each person in a variety of different ways and can range from very mild to severe. The functioning of the affected individual varies substantially depending on language abilities, level of intelligence, co-morbidity, composition of symptoms and access to services. Cognitive functioning, learning, attention and sensory processing are usually impaired.

DSM-IV states that the diagnosis of autism requires the presence of at least six symptoms, including a minimum of two measures of qualitative impairment in social interaction, one symptom of qualitative impairment in communication, and one symptom of restricted and repetitive behaviour. DMS-5 redefines diagnosis of ASD into two symptom domains: (i) social interaction and social communication deficits; and (ii) restricted, repetitive patterns of behaviour, interests or activities.

Co-morbid medical conditions are highly prevalent in ASDs. Co-morbid include anxiety and depression, seizures, attention deficits, aggressive behaviours, sleep problems, gastrointestinal disorders, epilepsy, mental retardation, intellectual disabilities and feeding difficulties.

The examples demonstrate that the compositions of the invention achieve a reduction in disease incidence and disease severity in an animal model of autism spectrum disorder and so they may be useful in the treatment or prevention of autism spectrum disorders.

Sodium butyrate is known to attenuate social behaviour deficits in an autism mouse model [26]. The examples demonstrate for the first time that a bacterial strain of the genus Fusicatenibacter can produce butyrate. Therefore, without wishing to be bound by any particular theory, the bacterial strain tested in the examples may reduce disease incidence and disease severity in an animal model of autism spectrum disorder by producing butyrate.

ASD is a central nervous system disorder that is partially triggered by environmental factors. Therefore, dysfunction of the microbiota-gut-brain axis may be responsible for development and persistence of ASDs. Accordingly, in preferred embodiments, the compositions of the invention are for use in treating or preventing autism spectrum disorders. In some embodiments, the compositions of the invention are for use in treating or preventing autism. In some embodiments, the autism is Pervasive Developmental Disorder (PDD). In another embodiment, the PDD is Asperger Syndrome, Childhood Autism/Autistic Disorder, Atyptical Autism and/or PDD—not otherwise specified. Accordingly, in some embodiments, the compositions of the invention are for use in treating or preventing autism spectrum disorders, autism, pervasive developmental disorder; Asperger Syndrome; Childhood Autism/Autistic Disorder, Atypical Autism and/or PDD—not otherwise specified.

The compositions of the invention may be useful for modulating the microbiota-gut-brain axis of a subject. Accordingly, in preferred embodiments the compositions of the invention are for use in preventing an ASD in a patient that has been identified as at risk of an ASD, or that has been diagnosed with an ASD at a prenatal or an early developmental stage; in childhood and/or in adulthood. The compositions of the invention may be useful for preventing the development of ASDs.

The compositions of the invention may be useful for managing or alleviating ASDs. Treatment or prevention of ASDs may refer to, for example, an alleviation of the severity of symptoms or a reduction in the frequency of exacerbations or the range of triggers that are a problem for the patient.

In preferred embodiments, the compositions of the invention prevent, reduce or alleviate at least one core symptom of ASDs.

In some embodiments, the compositions of the invention prevent, reduce or alleviate at least one of the two symptom domains of ASD classified in the DMS-5. In some embodiments, the compositions of the invention prevent, reduce or alleviate social interaction and/or social communication deficits. In some embodiments, the compositions of the invention prevent, reduce or alleviate restrictive, repetitive patterns of behaviour, interests or activities. In some embodiments, the compositions of the invention prevent, reduce or alleviate social interaction, social communication deficits and/or restrictive, repetitive patterns of behaviour, interests or activities.

In some embodiments, the compositions of the invention prevent, reduce or alleviate repetitive behaviour, stereotyped behaviour, compulsive behaviour, routine behaviour, sameness behaviour and restricted behaviour. In some embodiments, the compositions of the invention improve social awareness, social information processing, capacity for social communication, social anxiety/avoidance, and autistic preoccupations and traits in a subject with ASDs.

In some embodiments, the compositions of the invention prevent, reduce or alleviate additional symptoms associated with the core symptoms of ASDs. In some embodiments, the compositions of the invention prevent, reduce or alleviate irritability (including aggression, deliberate self-injury and temper tantrums), agitation, crying, lethargy, social withdrawal, stereotypic behaviour, hyperactivity, non-compliance, inappropriate speech, anxiety, depression, and/or over or under-controlled behaviour in a subject with ASDs. In some embodiments, the compositions of the invention improve cognitive functioning, learning, attention and/or sensory processing in a subject with ASD.

In other embodiments, the compositions of the invention improve secondary outcome measures in a subject with ASDs. In some embodiments, the secondary outcome measures include additional symptom and/or functional rating scales, behavioural scales and miscellaneous measures of interest.

In some embodiments, the compositions of the invention cause in a positive change in the diagnostic and/or symptomatic scale for the assessment of core symptoms of a subject with ASDs. In some embodiments, the diagnostic and/or symptomatic scale is the Autism Diagnostic Interview—Revised (ASI-R). In some embodiments, the diagnostic or symptomatic scale is the Autism Diagnostic Observation Schedule-Generic (ADOS-G) now ADOS-2. In other embodiments, the diagnostic or symptomatic scale is the Autism Diagnostic Interview Revised (ADI-R). In other embodiments, the diagnostic or symptomatic scale is the Diagnostic Interview for Social and Communication Disorders (DISCO). In yet other embodiments, the diagnostic or symptomatic scale is the Childhood Autism Rating Scale (CARS and CARS2).

In some embodiments, the compositions of the invention cause a positive change in generic measures of the efficacy endpoints of ASDs. In certain embodiments, the generic measures include, but are not limited to the Aberrant Behaviour Checklist (ABC), the Child Behaviour Checklist (CBCL), the Vineland-II Adaptive Behaviour Scales (VABS), the Social Responsiveness Scale (SRS), and/or the Repetitive Behaviour Scale—Revised (RBS-R).

In some embodiments, the compositions of the invention improve the Clinical Global Impression—Global Improvement (CGI-I) scale for assessing psychiatric and neurological disorders. In some embodiments, the compositions of the invention display a positive effect on global functioning of the subject with ASDs.

Additional scales would be known to a person skilled in the art. In some embodiments, the compositions of the invention would improve the outcome of diagnostic and/or symptomatic scales known to a person skilled in the art.

In certain embodiments, the compositions of the invention prevent, reduce or alleviate the incidence of comorbidities of ASDs. In some embodiments, the compositions of the invention prevent, reduce or alleviate the incidence of anxiety and depression, seizures, attention deficits, aggressive behaviours, sleep problems, gastrointestinal disorders (including irritable bowel syndrome (IBS)), epilepsy, mental retardation, intellectual disabilities and/or feeding difficulties. In certain embodiments, the compositions of the invention prevent, reduce or alleviate gastrointestinal comorbidities, such as abdominal pain, diarrhoea and flatulence.

In some embodiments, the compositions of the invention prevent, reduce or alleviate the symptoms of certain psychiatric and behavioural disorders that may present clinically with similarities to autism. Accordingly, in some embodiments, the compositions of the invention, prevent, reduce or alleviate attention deficit disorder (ADHD); affective/anxiety disorders; attachment disorders; oppositional defiant disorder (ODD); obsessive compulsive disorder (OCD) and/or psychoses including schizophrenia (cognitive impairment).

In some embodiments, the compositions of the invention are particularly effective at preventing, reducing or alleviating ASDs when used in combination with another therapy for treating ASDs. Such therapies include anti-psychotic, anti-anxiety and anti-depressant drugs. Such drugs include risperidone (Risperdal™); olanzapine (Zyprexa™); fluoxetine (Prozac™); sertraline (Zoloft™); fluvoxamine (Luvox™); clomipramine (Anafranil™); haloperidol (Haldol™); thioridazine; fluphenazine; chlorpromazine; ziprasidone (Geogon™); carbamazepine (Tegretol™); lamotrigine (Lamictal™); topiramate (Topomax™); valproic acid (Depakote™); methylphenidate (Ritalin™); diazepam (Valium™) and lorazepam (Ativan™).

The EMA Guidelines on the clinical development of medicinal products for the treatment of autism spectrum disorder state that, due to the heterogeneity of the diseases, it may not be possible to achieve a significant effect on all core symptoms with a single compound, and so short term efficacy has to be demonstrated on at least one core symptom. The live biotherapeutic strains used in the Examples have shown effective treatment of at least one core symptom of autistic spectrum disorder, so these strains and related Blautia strains are expected to be effective against human disease.

Obsessive Compulsive Disorder (OCD)

OCD is a heterogeneous, chronic and disabling disorder belonging to the anxiety disorders. According to the DSM-IV definition, the essential features of OCD are recurrent obsessions and/or compulsions (criterion A) that are severe and time consuming (more than one hour a day) or cause marked distress or significantly interfere with the subject's normal routine, occupational functioning, usual social activities or relationships (criterion C). As some point during the course of the disorder, the person has recognised that the obsessions or compulsions are excessive or unreasonable (criterion B).

Obsessions are defined as recurrent and persistent thoughts, impulses or images that are experienced as intrusive and inappropriate and cause marked anxiety or distress. The thoughts, impulses or images are not simply excessive worries about real-life problems, they are recognised by the patient as a product of his own mind (e.g. fear for contamination, symmetry obsession). The person attempts to ignore, suppress or neutralise the obsessions with some other thoughts or actions.

Compulsions are defined as repetitive behaviours (e.g. hand washing, ordering, hoarding, checking) or mental acts (e.g. praying, counting, repeating words silently) that the person feels driven to perform in response to an obsession or according to rules that must be applied rigidly.

OCD is often associated with co-morbidity rates of other psychiatric diseases including major depressive disorder, other anxiety disorders (generalised anxiety disorder, social anxiety disorder, panic disorder), substance abuse and eating disorders (anorexia and bulimia).

OCD is a psychiatric disorder that may develop or persist due to dysfunction of the microbiota-gut-brain axis. Accordingly, in preferred embodiments, the compositions of the invention are for use in treating or preventing OCD in a subject.

In certain embodiments, the compositions of the invention prevent, reduce or alleviate the essential symptomatic features of OCD. In certain embodiments, the compositions of the invention prevent, reduce or alleviate recurrent obsessions and/or compulsions in a subject. In certain embodiments, the obsessions are recurrent or persistent thoughts, impulses or images that are experiences as intrusive and inappropriate and cause marked anxiety or distress. In certain embodiments, the compulsions are repetitive behaviours that the subject feels driven to perform in response to an obsession or according to rules that must be applied rigidly.

In certain embodiments, the compositions of the invention improve symptoms of OCD in a subject accordingly to the Y-BOCS and/or the NIMH-OC diagnostic and/or symptomatic scales. In some embodiments, the Y-BOCS scale is used to monitor improvement of primary endpoints. In some embodiments, the NIMH-OC scale is used to monitor improvement of secondary parameters.

In some embodiments, the compositions of the invention improve the Clinical Global Impression—Global Improvement (CGI-I) scale for assessing psychiatric and neurological disorders. In some embodiments, the compositions of the invention display a positive effect on global social functioning (relationships, work, etc.) of the subject with ASDs. In some embodiments, the global scale is the Sheehan disability scale.

In preferred embodiments, the compositions of the invention prevent, reduce or alleviate at least one comorbidity of OCD. The comorbidities of OCD include major depressive disorder, other anxiety disorders (generalised anxiety disorder, social anxiety disorder, panic disorder), substance abuse and eating disorders (anorexia and bulimia) Gilles de la Tourette syndrome, ADHD (Attention-Deficit/Hyperactivity Disorder) and developmental disorders.

In some embodiments, the compositions of the invention are particularly effective at preventing, reducing or alleviating OCD when used in combination with another therapy for treating OCD. Such therapies include serotonin and dopamine reuptake inhibitors; clomipramine and anti-psychotics.

Major Depressive Disorder (MDD)

MDD is associated with substantial psychosocial dysfunction and high individual mental strain as well as with excess morbidity and mortality (the risk of suicide is considerable). The term major depressive disorder encompasses clinical depression, major depression, unipolar depression, unipolar disorder, recurrent depression and simply depression. The term major depressive disorder covers mood disorders; dysthymia; chronic depression; seasonal affective disorder and borderline personality disorder.

According to the DMS-5 criteria, MDD symptoms include a depressed mood, or loss of interest or pleasure in daily activities for more than two weeks; and impaired social, occupational and educational function. Specific symptoms, at least five of the following nine, present nearly every day: depressed mood or irritable most of the day; decreased interest or pleasure in most activities, most of each day; significant weight change or change in appetite; change in sleep (insomnia or hypersomnia); change in activity (psychomotor agitation or retardation); fatigue or loss of energy; guilt or worthlessness (feelings of worthlessness or excessive or inappropriate guilt); reduced concentration (diminished ability to think or concentrate, or more indecisiveness; and suicidality (thoughts of death or suicide, or subject has a suicide plan). In addition, MDD is associated with anxiety symptoms including irrational worry; preoccupation with unpleasant worries; trouble relaxing and/or feeling tense. MDD episodes can be mild, moderate or severe.

MDD episodes are often associated with comorbidity with other psychiatric disorders or with somatic disorders like Parkinson's disease, Alzheimer's disease, cerebrovascular disorders, cancer and chronic pain syndromes. MDD is frequently associated with a wide spectrum of other mental disorders as comorbidities including generalised anxiety disorder; anxiety disorder; substance use disorders; post-traumatic stress disorder (PTSD); personality disorders; pain; stress; irritable bowel syndrome; insomnia; headaches and interpersonal problems.

Major depressive disorder is a psychiatric disorder that may develop or persist due to dysfunction of the microbiota-gut-brain axis. Accordingly, in preferred embodiments, the compositions of the invention are for use in treating or preventing MDD in a subject.

In certain embodiments, the compositions of the invention are for use in treating or preventing acute major depressive episodes and/or the prevention of new episodes (recurrence prevention). In certain embodiments, the compositions of the invention prevent, reduce or alleviate the occurrence of mild, moderate or severe MDD episodes.

In certain embodiments, the compositions of the invention prevent, reduce or alleviate one or more of the symptoms of MDD as classified by the DMS-5 criteria listed herein. In a preferred embodiment, the compositions of the invention prevent, reduce or alleviate a depressed mood in a subject. In a preferred embodiment, the compositions of the invention prevent, reduce or alleviate a decreased interest or pleasure in most activities in a subject. In some embodiments, the compositions of the invention reduce the occurrence of symptoms of MDD within a 2-week period.

In some embodiments, the compositions of the invention improve the symptoms of MDD according to a symptomatic or diagnostic scale. Such scales for assessing symptomatic improvement include the Hamilton Rating Scale of Depression (HAMD) and the Montgomery Asberg Depression Rating Scale.

In addition, the Zung Self-Rating Depression Scale (SDS) and Zung Self-Rating Anxiety Scale (SAS) are also suitable symptomatic improvement scales.

In some embodiments, the compositions of the invention improve the Clinical Global Impression—Global Improvement (CGI-I) scale for assessing psychiatric and neurological disorders. In some embodiments, the compositions of the invention display a positive effect on global social and occupational functioning of the subject with MDD.

In certain embodiments, the compositions of the invention are for use in treating or preventing treatment resistant MDD.

In preferred embodiments, the compositions of the invention prevent, reduce or alleviate at least one comorbidity of MDD. The comorbidities of MDD include generalised anxiety disorder; anxiety disorder; substance use disorders; post-traumatic stress disorder (PTSD); personality disorders; pain; stress; IBS; insomnia; headaches and interpersonal problems.

In some embodiments, the compositions of the invention are particularly effective at preventing, reducing or alleviating MDD when used in combination with another therapy for treating MDD. Such therapies include antidepressants, augmentation strategies (e.g. combination therapy, lithium and other mood stabilizers, thyroid hormones and atypical antipsychotics) or even second generation antipsychotics.

Anxiety Disorders

Anxiety disorders are a group of mental disorders characterised by feelings of anxiety and fear. There are a number of anxiety disorders including generalised anxiety disorder (GAD); specific phobia; social anxiety disorder; separation anxiety disorder; agoraphobia; panic disorder and selective mutism.

GAD is diagnosed according to DMS-5 in six criterion. The first criterion is too much anxiety or worry over more than six months wherein the anxiety or worry is present most of the time in regards to many activities. The second criterion is that the subject is unable to manage the symptoms of the first criterion. The third criterion is that at least three (one in children) of the following occurs: restlessness; tires easily; problems concentrating; irritability; muscle tension and problems with sleep. The final three criterion are that the symptoms results in significant social, occupational and functional impairment; the symptoms are not due to medications, drugs, or other physical health problems; and the symptoms do not fit better with another psychiatric problem such as panic disorder. All other anxiety disorders may be considered as differential diagnoses of GAD.

GAD is frequently associated with a wide spectrum of other mental disorders as comorbidities including depression; substance use disorders; stress; IBS; insomnia; headaches; pain; cardiac events; interpersonal problems and ADHD.

Anxiety disorders are psychiatric disorders that may develop or persist due to dysfunction of the microbiota-gut-brain axis. Accordingly, in preferred embodiments, the compositions of the invention are for use in treating or preventing anxiety disorders in a subject. In certain embodiments, the anxiety disorder is generalised anxiety disorder (GAD); specific phobia; social anxiety disorder; separation anxiety disorder; agoraphobia; panic disorder and selective mutism.

In certain embodiments, the compositions of the invention prevent, reduce or alleviate one or more of the symptoms of GAD in a subject as classified by the DMS-5 criteria listed herein. According to DMS-5, the same symptoms are associated with other anxiety disorders. Therefore, in certain embodiments, the compositions of the invention prevent, reduce or alleviate one or more of the symptoms of anxiety disorders in a subject. In preferred embodiments, the compositions of the invention prevent, reduce or alleviate the anxiety or worry of the subject. In certain embodiments, the compositions of the invention reduce the occurrence of symptoms within a six month period. In certain embodiments, the composition of the invention prevents, reduces or alleviates restlessness; fatigue; loss of concentration; irritability; muscle tension; and/or problems with sleep. In some embodiments, the compositions of the invention prevent, reduce or alleviate social, occupational and functional impairment associated with anxiety disorders.

In some embodiments, the compositions of the invention improve the symptoms of anxiety disorders according to a symptomatic or diagnostic scale. In certain embodiments, the scale for assessing symptomatic improvement includes the Hamilton Anxiety Rating Scale (HAM-A). In some embodiments, the HAM-A total scale is used to assess primary endpoint. In other embodiments, the HAM-A psychic anxiety factor may be useful as a secondary endpoint.

In some embodiments, the compositions of the invention improve the Clinical Global Impression—Global Improvement (CGI-I) scale for assessing psychiatric and neurological disorders. In some embodiments, the compositions of the invention display a positive effect on global social, occupational and functional impairment of the subject with anxiety disorder. In some embodiments, the global scale is the Sheehan disability scale.

In preferred embodiments, the compositions of the invention prevent, reduce or alleviate at least one comorbidity of GAD and anxiety disorders. The comorbidities of GAD include depression; substance use disorders; stress; IBS; insomnia; headaches; pain; cardiac events; interpersonal problems and ADHD.

In some embodiments, the compositions of the invention are particularly effective at preventing, reducing or alleviating anxiety disorders when used in combination with another therapy for treating anxiety disorders. Such therapies include selective serotonin reuptake inhibitors (venlafaxine, duloxetine, escitalopram and paroxetine); benzodiazepines (alprazolam, lorazepam and clonazepam); pregabalin (Lyrica®) and gabapentin (Neurontin®); serotonin receptor partial agonists (buspirone and tandospirone); atypical serotonergic antidepressants (such as imipramine and clomipramine); monoamine oxidase inhibitors (MAOIs) (such as moclobemide and phenelzine); hydroxyzine; propranolol; clonidine; guanfacine and prazosin.

Post-Traumatic Stress Disorder (PTSD)

PTSD is a severe and disabling disorder, an essential feature of which is the inclusion of a traumatic event as a precipitating factor of this disorder.

The symptoms of PTSD are grouped into four main clusters according to the DMS-V criteria: (i) intrusion: examples include nightmares, unwanted thoughts of the traumatic events, flashbacks, and reacting to traumatic reminders with emotional distress or physiological reactivity; (ii) avoidance: examples include avoiding triggers for traumatic memories including places, conversations, or other reminders; (iii) negative alterations in cognitions and mood: examples include distorted blame of self or others for the traumatic event, negative beliefs about oneself or the world, persistent negative emotions (e.g., fear, guilt, shame), feeling alienated, and constricted affect (e.g., inability to experience positive emotions); (iv) alterations in arousal and reactivity: examples include angry, reckless, or self-destructive behaviour, sleep problems, concentration problems, increased startle response, and hypervigilance.

Symptoms that resolve within 4 weeks of the traumatic event meet the criteria for an Acute Stress Disorder. The DSM distinguishes between acute (duration of symptoms for less than three months) and chronic PTSD (duration of symptoms longer than 3 months). If the symptoms begin more than 6 months after the stressor, the disorder is defined as delayed onset PTSD.

PTSD carries high comorbidities with major depressive disorder and substance use disorders.

PTSD is a psychiatric disorder that may develop or persist due to dysfunction of the microbiota-gut-brain axis. Accordingly, in preferred embodiments, the compositions of the invention are for use in treating or preventing PTSD in a subject. According to a similar pathogenesis, in certain embodiments, the compositions of the invention are for use in treating or preventing stress disorders. In certain embodiments, the compositions of the invention treat acute stress disorder. In some embodiments, the compositions of the invention treat acute and/or chronic PTSD. In some embodiments, the compositions of the invention treat delayed onset PTSD.

In certain embodiments, the compositions of the invention prevent, reduce or alleviate one or more of the symptoms of PTSD (or stress disorder) in a subject as classified by the DMS-5 criteria listed herein.

In preferred embodiments, the compositions of the invention prevent, reduce or alleviate intrusive thoughts in a subject with PTSD. In preferred embodiments, the compositions of the invention prevent, reduce or alleviate avoidance behaviour in a subject with PTSD. In preferred embodiments, the compositions of the invention prevent, reduce or alleviate negative alterations in cognitions and mood in a subject with PTSD. In preferred embodiments, the compositions of the invention prevent alterations in arousal and reactivity in a subject with PTSD.

In some embodiments, the compositions of the invention improve the symptoms of PTSD and stress disorders according to a symptomatic or diagnostic scale. In certain embodiments, the scale for assessing symptomatic improvement is the Clinical-Administered PTSD (CAPS) scale.

In some embodiments, the compositions of the invention improve the Clinical Global Impression—Global Improvement (CGI-I) scale for assessing psychiatric and neurological disorders. In some embodiments, the compositions of the invention display a positive effect on global social, occupational and functional impairment of the subject with PTSD and stress disorders. In some embodiments, the global scale is the Sheehan disability scale.

In preferred embodiments, the compositions of the invention prevent, reduce or alleviate at least one comorbidity of PTSD and stress disorders. The comorbidities of PTSD and stress disorders include MDD, substance use disorders; stress and anxiety.

In some embodiments, the compositions of the invention are particularly effective at preventing, reducing or alleviating PTSD and stress disorders when used in combination with another therapy for treating PTSD and stress disorders. Such therapies include serotoninergic agents, tricyclic antidepressants, mood stabilisers, adrenergic inhibiting agents, antipsychotics, benzodiazepines, sertraline (Zoloft™), fluoxetine (Prozac™) and/or paroxetine (Paxil™)

Schizophrenia Spectrum and Psychotic Disorders

These diseases affect a subject's ability to think clearly, make good judgements, respond emotionally, communicate effectively, understand reality, and behave appropriately. Psychotic diseases include schizophrenia (symptoms listed below); schizoaffective disorder (the subject has symptoms of both schizophrenia and a mood disorder, such as depression or bipolar disorder); schizophreniform disorder (displays the symptoms of schizophrenia, but the symptoms last for a shorter time: between 1 and 6 months); brief psychotic disorder (subjects display a sudden, short period of psychotic behaviour, often in response to a very stressful event, such as a death in the family—recovery is usually less than a month); delusional disorder (delusions last for at least 1 month); shared psychotic disorder; substance-induced psychotic disorder; psychotic disorder due to another medical condition; paraphrenia (displaying symptoms similar to schizophrenia and starting late in life, when people are elderly). The most well-known psychotic disorder is schizophrenia and the majority of psychotic disorders display similar symptoms to schizophrenia.

Schizophrenia is a severe psychiatric disease with a heterogeneous course and symptom profile. Schizophrenia presents clinically with so-called positive and negative symptoms. The positive symptoms include delusions, hallucinations, disorganised speech, and disorganised or catatonic behaviours. Negative symptoms include affective flattening, restriction in the fluency and productivity of thought and speech and in the initiation of goal directed behaviour. The positive symptoms appear to reflect an excess or distortion of normal functions, whereas negative symptoms appear to reflect a diminution or loss of normal function. In addition, cognitive deficits (defects of working memory, information processing, attention/vigilance, learning, reasoning and social cognition) are common. Cognitive deficits generally show poor improvement with current antipsychotic treatment. Schizophrenic patients also suffer from mood symptoms. Besides these predominant symptoms, schizophrenia is associated with a comorbidity with other psychiatric symptoms such as manic and depressive symptoms, anxiety or obsessive-compulsive symptoms, substance abuse and dependence, and personality disorder.

According to the DMS-5, for the diagnosis of schizophrenia, a subject must have at least two of the following symptoms: delusions; hallucinations; disorganised speech; disorganised or catatonic behaviour and negative symptoms. At least one of the symptoms must be the presence of delusions, hallucinations or disorganised speech. Continuous signs of disturbance must persist for at least 6 months, during which the subject must experience at least 1 month of active symptoms, with social or occupational deterioration problems occurring over a significant amount of time.

Schizophrenia spectrum and psychotic disorders are psychiatric disorders that may develop or persist due to dysfunction of the microbiota-gut-brain axis. Therefore, in preferred embodiments, the compositions of the invention are for use in treating or preventing schizophrenia spectrum and/or psychotic disorders in a subject. In certain embodiments, the schizophrenia spectrum and psychotic disorder is selected from schizophrenia; schizoaffective disorder; schizophreniform disorder; brief psychotic disorder; delusional disorder; shared psychotic disorder; substance-induced psychotic disorder; psychotic disorder due to another medical condition and paraphrenia. In preferred embodiments, the compositions of the invention are for use in treating or preventing schizophrenia. In certain embodiments, the schizophrenia is selected from paranoid, disorganised, catatonic, undifferentiated and residual schizophrenia.

In certain embodiments, the compositions of the invention prevent, reduce or alleviate one or more of the symptoms of schizophrenia in a subject as classified by the DMS-5 criteria listed herein. These embodiments apply to the prevention, reduction or alleviation of symptoms of other schizophrenia spectrum and psychotic disorders. In certain embodiments, the compositions of the invention prevent, reduce or alleviate negative symptoms of schizophrenia. In certain embodiments, the compositions of the invention prevent, reduce or alleviate positive symptoms of schizophrenia. In certain embodiments, the compositions of the invention prevent, reduce or alleviate negative and positive symptoms of schizophrenia. In preferred embodiments, the compositions of the invention prevent, reduce or alleviate delusions, hallucinations, disorganised speech, and disorganised or catatonic behaviours in a subject with schizophrenia. In preferred embodiments, the compositions of the invention prevent, reduce or alleviate affective flattening, restriction in the fluency and productivity of thought and speech and in the initiation of goal directed behaviour in a subject with schizophrenia. In preferred embodiments, the compositions of the invention prevent, reduce or alleviate the cognitive defects and/or mood disorders in a subject with schizophrenia.

In certain embodiments, the compositions of the invention reduce the occurrence of positive and/or negative symptoms of schizophrenia in a subject within a 6 month period. In certain embodiments, the compositions of the invention improve social and/or occupational functionality in a subject with schizophrenia spectrum or psychotic disorder.

In some embodiments, the compositions of the invention improve the symptoms of schizophrenia spectrum or psychotic disorders according to a symptomatic or diagnostic scale. In certain embodiments, the scale for assessing symptomatic improvement is the Positive and Negative Symptom Scale (PANSS) and Brief Psychiatric Rating Scale (BPRS). In certain embodiments, the Scale for Assessment of Negative Symptoms (SANS) is used.

In some embodiments, the compositions of the invention improve the Clinical Global Impression—Global Improvement (CGI-I) scale for assessing psychiatric and neurological disorders. In some embodiments, the compositions of the invention display a positive effect on global social and occupational impairment of the subject with schizophrenia spectrum or psychotic disorders.

In preferred embodiments, the compositions of the invention prevent, reduce or alleviate at least one comorbidity of schizophrenia spectrum or psychotic disorder. In certain embodiments, the comorbidity is as manic and depressive symptoms, anxiety or obsessive-compulsive symptoms, substance abuse and dependence, and personality disorder.

In certain embodiments, the compositions of the invention are for use in treating or preventing treatment resistant of refractory schizophrenia.

In some embodiments, the compositions of the invention are particularly effective at preventing, reducing or alleviating schizophrenia spectrum or psychotic disorders when used in combination with another therapy for treating PTSD and stress disorders. In certain embodiments, such therapies include first generation antipsychotics including chlorpromazine, fluphenazine, haloperidol and/or perphenazine. In certain embodiments, such therapies include second generation therapies including aripiprazole (Abilify™); asenapine (Saphris™); brexpiprazole (Rexulti™); cariprazine (Vraylar™); clozapine (Clozaril™); iloperidone (Fanapt™); lurasidone (Latuda™); olanzapine (Zyprexa™); paliperidone (Invega); quetiapine (Seroquel™); risperidone (Risperdal™); ziprasidone (Geodon™)

Bipolar Disorder

Bipolar disorder in general is a chronic disease. Mania is the cardinal symptom of bipolar disorder. There are several types of bipolar disorder based upon the specific duration and pattern of manic and depressive episodes. In DMS-5, a distinction is made between bipolar I disorder, bipolar II disorder, cyclothymic disorder, rapid-cycling bipolar disorder and bipolar disorder NOS.

According to the DSM, mania is a distinct period of abnormally and persistently elevated, expansive, or irritable mood. The episode must last a week, and the mood must have at least three of the following symptoms: high self-esteem; reduced need for sleep; increase rate of speech; rapid jumping of ideas; easily distracted; an increased interest in goals or activities; psychomotor agitation; increased pursuit of activities with a high risk of danger.

Bipolar I disorder involves one or more manic or mixed (mania and depression) episodes and at least one major depressive episode (see above for symptoms of MDD episodes). Bipolar II disorder has one or more major depressive episodes accompanied by at least one hypomanic episode. There are no manic or mixed episodes. Hypomania is a lesser form of mania. The symptoms are responsible for significant social, occupational and functional impairments. Cyclothymia is characterized by changing low-level depression along with periods of hypomania. The symptoms must be present for at least two years in adults or one year in children before a diagnosis can be made. Symptom free periods in adults and children last no longer than two months or one month, respectively. Rapid cycling bipolar disorder is a severe form of bipolar disorder. It occurs when a person has at least four episodes of major depression, mania, hypomania, or mixed states within a year. Not-otherwise specified (NOS) bipolar disorder classified bipolar symptoms that do not clearly fit into other types. NOS is diagnosed when multiple bipolar symptoms are present but not enough to meet the label for any of the other subtypes.

Bipolar disorder is associated with the following comorbidities: ADHD; anxiety disorders; substance disorders; obesity and metabolic syndrome.

Bipolar disorder is a psychiatric disorder that may develop or persist due to dysfunction of the microbiota-gut-brain axis. Therefore, in preferred embodiments, the compositions of the invention are for use in treating or preventing bipolar disorder in a subject. In certain embodiments, the bipolar disorder is bipolar I disorder. In certain embodiments, the bipolar disorder is bipolar II disorder. In certain embodiments, the bipolar disorder is cyclothymic disorder. In certain embodiments, the bipolar disorder is rapid-cycling bipolar disorder. In certain embodiments, the bipolar disorder is bipolar disorder NOS.

In preferred embodiments, the compositions of the invention prevent, reduce or alleviate one or more of the symptoms of bipolar disorder in a subject. In certain embodiments, the compositions of the invention prevent, reduce or alleviate the occurrence of manic episodes in a subject. In certain embodiments, the compositions of the invention prevent, reduce or alleviate the occurrence of an abnormally and persistently elevated, expansive, or irritable mood. In certain embodiments, the compositions of the invention prevent, reduce or alleviate one or more of the following symptoms: high self-esteem; reduced need for sleep; increase rate of speech; rapid jumping of ideas; easily distracted; an increased interest in goals or activities; psychomotor agitation; increased pursuit of activities with a high risk of danger. In certain embodiments, the compositions of the invention prevent, reduce or alleviate the occurrence of one or more manic or mixed episodes in a subject. In certain embodiments, the compositions of the invention reduce the occurrence of at least one major depressive episode in a subject. In certain embodiments, the compositions of the invention prevent, reduce or alleviate the occurrence of at least one major depressive episode accompanied by at least one hypomanic episode.

In preferred embodiments, the compositions of the invention treat the acute phase of bipolar disorder and/or prevent the occurrence of further episodes. In certain embodiments, the compositions of the invention treat the acute phase of manic/depressive episodes in a subject with bipolar disorder and prevent occurrence of further manic/depressive episodes.

In some embodiments, the compositions of the invention improve the symptoms of bipolar disorder according to a symptomatic or diagnostic scale. In certain embodiments, the scale for assessing symptomatic improvement of manic episodes is the Manic State Rating Scale and the Young Mania Rating Scale. In certain embodiments, the scale is the Bech-Rafaelsen Mania Scale (BRMAS). In certain embodiments, scales for assessing symptomatic improvement of the switch from manic to depressive episodes include the Hamilton Depression Rating Scale, the Montgomery-Asberg Rating Scale, and the Bech-Rafaelsen Depression Scale.

In some embodiments, the compositions of the invention improve the Clinical Global Impression—Global Improvement (CGI-I) scale for assessing psychiatric and neurological disorders. In some embodiments, the compositions of the invention display a positive effect on global social, occupational and functional impairments of the subject with bipolar disorder.

In preferred embodiments, the compositions of the invention prevent, reduce or alleviate at least one comorbidity of bipolar disorder. In certain embodiments, the comorbidity is selected from ADHD, anxiety disorders, substance disorder, obesity and metabolic syndrome.

In certain embodiments, the compositions of the invention are for use in treating or preventing manic-depressive illness and bipolar disorder unresponsive to lithium and divalproex.

In some embodiments, the compositions of the invention are particularly effective at preventing, reducing or alleviating bipolar disorder when used in combination with another therapy for treating bipolar disorder. In certain embodiments, such therapies include lithium carbonate, anticonvulsant drugs (including valproate, divalproex, carbamazepine and lamotrigine) and antipsychotic drugs (including aripiprazole, olanzapine, quetiapine and risperidone).

Intestinal Permeability

Bacteria can modulate signalling of the microbiota-gut-brain axis by modulating the levels of intestinal permeability. The intestinal barrier is a complex multilayer system that separates the internal milieu from the luminal environment, consisting of an external “physical” barrier and an inner “functional” immunological barrier. The intestinal barrier prevents the entry of antigens and microorganisms into the body while allowing exchange of molecules between host and environment and the absorption of nutrients. Intestinal gut permeability is defined as a functional feature of the intestinal barrier at given sites, measurable by analysing the flux rate if inert molecules across the intestinal wall [27].

Diseases related to intestinal gut permeability, in addition to several of the CNS disorders discussed herein, include intestinal diseases; such as gastric ulcers, infectious diarrhea, irritable bowel syndrome; functional GI diseases, inflammatory bowel disease, celiac disease and cancer (esophagus, colorectal) and extraintestinal diseases; such as food allergies, infections (e.g. respiratory), acute inflammation (sepsis, systemic inflammatory response syndrome (SIRS), multiple organ failure), obesity, metabolic diseases (e.g. s such as NAFLD and NASH, type 2 diabetes mellitus or cardiovascular diseases) and chronic inflammation (e.g. arthritis). These diseases are characterized by inflammation that might be triggered by the translocation of components from the lumen into the body [27].

The inventors have demonstrated that bacterial strains of the genus Fusicatenibacter can decrease intestinal permeability in the gut. Butyrate is known to play a role in maintaining the intestinal barrier. A reduction in butyrate causes tight junction lesions and impaired intestinal permeability [27]. The examples demonstrate that the bacterial strains of the invention can increase butyrate production. Therefore, the compositions of the invention are useful for reducing intestinal permeability.

The bacterial strains of the invention are therefore particularly useful in the improvement of gut barrier function and thus the treatment or prevention of diseases which are associated with increased intestinal permeability. The bacterial strains of the invention are also useful in the treatment or prevention of diseases which are characterised by altered intestinal permeability. The compositions of the invention are be useful for decreasing intestinal permeability in a subject, in particular by inducing butyrate production and/or by increasing butyrate levels in the gastrointestinal tract. The examples demonstrate for the first time that bacterial strains from the genus Fusicatenibacter can produce butyrate.

In embodiments of the invention, the compositions of the invention are for improving gut barrier function, for example in subjects identified as having impaired gut barrier function. This effect of the compositions of the invention provides a new clinical situation as subjects identified as having impaired gut barrier function comorbid with other conditions, particularly those associated with increased intestinal permeability may be more likely to respond to treatment using compositions of the present invention.

Therefore, the compositions of the invention may be useful for treating or preventing gastric ulcers, infectious diarrhea, irritable bowel syndrome, functional GI diseases, inflammatory bowel disease, celiac disease and cancer (esophagus, colorectal), food allergies, infections (e.g. respiratory), acute inflammation (sepsis, systemic inflammatory response syndrome (SIRS), multiple organ failure), obesity, metabolic diseases (e.g. NAFLD and NASH, type 2 diabetes mellitus or cardiovascular diseases) and chronic inflammation (e.g. arthritis).

The compositions of the invention, therefore, may be particularly effective at preventing or delaying the onset or progression of diseases which are associated with increased or altered intestinal permeability. In certain embodiments, the compositions of the invention are for use in delaying the onset or progression of a disease which is associated with increased or altered intestinal permeability. The prevention of diseases which are associated with increased or altered intestinal permeability after administration of compositions of the invention can be measured relative to the disease state observed in the patient before treatment, a healthy individual or an individual with a more severe form of the disease which is associated with increased or altered intestinal permeability.

Short Chain Fatty Acid (SCFA) Production

Short chain fatty acids (SCFAs), such as acetate (C2), propionate (C3) and butyrate (C4), are metabolic products of anaerobic bacteria fermentation in the intestine. These SCFAs are key mediators of the beneficial effects elicited by the gut microbiome and are suggested to be the link between the microbiota and the host tissues. SCFAs, mainly butyrate, can modulate the production of inflammatory mediators (including TNF-α, IL-6 and IL-10) by macrophages. Due to their ability to regulate the inflammatory response, the therapeutic application of SCFAs in inflammatory conditions, such as inflammatory bowel disease, sepsis and acute lung injury (ALI), has been proposed [28]. The inventors have newly identified that bacterial strains from the genus Fusicatenibacter can produce butyrate. Without wishing to be bound by any particular theory, the composition of the invention may be particularly effective at treating or preventing inflammatory conditions, such as inflammatory bowel disease, sepsis and acute lung injury (ALI), by modulating the production of inflammatory mediators, such as TNF-α, IL-6 and IL-10.

SCFAs have also been implicated in modulating metabolic and cardiovascular health. SCFAs have been shown to modulate systolic blood pressure and diastolic blood pressure, for example studies have shown that butyrate can reduce diastolic blood pressure through a reduction in inflammation. Additionally, SCFAs have been suggested to protect against diet-induced obesity by reducing appetite and energy intake and by modulating glucose homeostasis [29]. The trimethylamine-N-oxide (TMAO) pathway has been found to link the gut microbiota and cardiovascular diseases, such as atherosclerosis [30].

The examples demonstrate that bacteria from the genus Fusicatenibacter can increase SCFA production including butyrate. Therefore, the compositions of the invention may be useful for treating or preventing inflammatory diseases, such as inflammatory bowel disease, sepsis and acute lung injury (ALI).

Furthermore, the compositions of the invention may be useful for treating or preventing metabolic diseases, such as obesity and diabetes and cardiovascular diseases, such as atherosclerosis, hypertension, coronary artery disease, angina and myocardial infarction, heart arrhythmia, heart failure, valvular heart disease, Pericardial disease, stroke, hypertensive heart disease, rheumatic heart disease, cardiomyopathy, congenital heart disease, carditis, aortic aneurysms, peripheral artery disease, thromboembolic disease, and venous thrombosis.

Modes of Administration

Preferably, the compositions disclosed herein are to be administered to the gastrointestinal tract in order to enable delivery to and/or partial or total colonisation of the intestine with the bacterial strain of the invention. In other words, the bacteria may have colonised some or all of the gastrointestinal tract and/or such colonisation may be transient or permanent.

More specifically, in some embodiments, the “total colonisation of the intestine” means that bacteria have colonised all parts of the intestine (i.e. the small intestine, large intestine and rectum). Additionally or alternatively, the term “total colonisation” means that the bacteria engraft permanently in the some or all parts of the intestine.

In some embodiments, “partial colonisation of the intestine” means that bacteria have colonised some but not all parts of the intestine. Additionally or alternatively, the term “partial colonisation” means that the bacteria engraft transiently in some or all parts of the intestine.

The transience of engraftment can be determined by assessing (e.g. in a fecal sample) the abundance of the bacterial strain of the invention periodically (e.g. daily) following the end of a dosing interval to determine the washout period, i.e. the period between conclusion of the dosing interval and there being no detectable levels of the bacterial strain of the invention present. In embodiments of the invention, the washout period is 14 days or less, 12 days or less, 10 days or less, 7 days or less, 4 days or less, 3 days or less, 2 days or less or 1 day or less.

In embodiments of the invention, the bacteria of the present invention engraft transiently in the large intestine.

Generally, the compositions of the invention are administered orally, but they may be administered rectally, intranasally, or via buccal or sublingual routes.

In certain embodiments, the compositions of the invention may be administered as a tablet, a foam, as a spray or a gel.

In certain embodiments, the compositions of the invention may be administered as a suppository, such as a rectal suppository, for example in the form of a theobroma oil (cocoa butter), synthetic hard fat (e.g. suppocire, witepsol), glycero-gelatin, polyethylene glycol, or soap glycerin composition.

In certain embodiments, the compositions of the invention are administered to the gastrointestinal tract via a tube, such as a nasogastric tube, orogastric tube, gastric tube, jejunostomy tube (J tube), percutaneous endoscopic gastrostomy (PEG), or a port, such as a chest wall port that provides access to the stomach, jejunum or other suitable access ports.

The compositions of the invention may be administered once, or they may be administered sequentially as part of a treatment regimen. In certain embodiments, the compositions of the invention are to be administered daily (either once or several times).

In certain embodiments, the compositions of the invention are administered regularly, such as daily, every two days, or weekly, for an extended period of time, such as for at least one week, two weeks, one month, two months, six months, or one year.

In some embodiments the compositions of the invention are administered for 7 days, 14 days, 16 days, 21 days or 28 days or no more than 7 days, 14 days, 16 days, 21 days or 28 days. For example, in some embodiments the compositions of the invention are administered for 16 days.

In certain embodiments of the invention, treatment according to the invention is accompanied by assessment of the patient's gut microbiota. Treatment may be repeated if delivery of and/or partial or total colonisation with the strain of the invention is not achieved such that efficacy is not observed, or treatment may be ceased if delivery and/or partial or total colonisation is successful, and efficacy is observed.

In certain embodiments, the composition of the invention may be administered to a pregnant animal, for example a mammal such as a human in order prevent diseases, disorders or conditions from developing in her child in utero and/or after it is born. For example, the composition of the invention may be administered to a pregnant animal in order prevent central nervous system diseases or disorders from developing.

The compositions of the invention may be administered to a patient that has been diagnosed with a disease, a disorder or a condition, such as a central nervous system disease or disorder, or that has been identified as being at risk of disease, a disorder or a condition. The compositions may also be administered as a prophylactic measure to prevent the development of disease, a disorder or a condition in a healthy patient.

The compositions of the invention may be administered to a patient that has been diagnosed with autism. They may also be administered to a patient that has been identified as being at risk of autism. The compositions may also be administered as a prophylactic measure to prevent the development of autism in a healthy patient.

The compositions of the invention may be administered to a patient that has been identified as having an abnormal gut microbiota. For example, the patient may have reduced or absent colonisation by Fusicatenibacter, and in particular Fusicatenibacter saccharivorans.

The compositions of the invention may be administered as a food product, such as a nutritional supplement.

Generally, the compositions of the invention are for the prevention or treatment of humans, although they may be used to treat animals including monogastric mammals such as poultry, pigs, cats, dogs, horses or rabbits. The compositions of the invention may be useful for enhancing the growth and performance of animals. If administered to animals, oral gavage may be used.

In some embodiments, the subject to whom the composition is to be administered is an adult human. In some embodiments, the subject to whom the composition is to be administered is a child human. In some embodiments, the subject to whom the composition is to be administered is an infant human.

Compositions

The compositions of the invention comprise bacteria. The inventors have identified the surprising ability of bacteria from the genus Fusicatenibacter to treat inflammatory central nervous system diseases or disorders. However, in order for bacteria from the genus Fusicatenibacter to exert their beneficial effect they need to be effectively delivered alive and/or viable to the small intestine. In general, a composition of the invention therefore does not comprise inactivated bacteria of the species Fusicatenibacter, in particular heat-inactivated bacteria of the species Fusicatenibacter.

The invention provides compositions which are formulated to prevent the bacteria from being degraded or absorbed in the upper digestive tract and being unable to exert their effect. For example, the compositions may comprises oxygen scavengers and/or prebiotic substrates, such as vitamin C and non-digestible carbohydrates.

In addition, the composition can be enterically formulated to ensure that the bacteria are not degraded on route to the small intestine.

Therefore, in preferred embodiments, the composition of the invention is encapsulated to enable delivery of the bacterial strain to the intestine. Encapsulation protects the composition from degradation until delivery at the target location through, for example, rupturing with chemical or physical stimuli such as pressure, enzymatic activity, or physical disintegration, which may be triggered by changes in pH. Any appropriate encapsulation method may be used. Exemplary encapsulation techniques include entrapment within a porous matrix, attachment or adsorption on solid carrier surfaces, self-aggregation by flocculation or with cross-linking agents, and mechanical containment behind a microporous membrane or a microcapsule. Guidance on encapsulation that may be useful for preparing compositions of the invention is available in, for example, references [31] and [32].

In preferred embodiments of the invention, the composition is formulated in freeze-dried form. For example, the composition of the invention may comprise granules or gelatin capsules, for example hard gelatin capsules, comprising a bacterial strain of the invention. Preferably, the composition of the invention comprises lyophilised bacteria. Lyophilisation of bacteria is a well-established procedure and relevant guidance is available in, for example, references [33-35]. The examples demonstrate that lyophilised compositions are particularly effective.

Alternatively, the composition of the invention may comprise a live, active bacterial culture. The examples demonstrate that cultures of the bacteria of the invention are therapeutically effective.

The bacterial strain in the composition of the invention may not have been inactivated, for example, may not have been heat-inactivated. The bacterial strain in the composition of the invention may not have been killed, for example, not been heat-killed. The bacterial strain in the composition of the invention may not have been attenuated, for example, not been heat-attenuated. For example, the bacterial strain in the composition of the invention may not have been killed, inactivated and/or attenuated. For example, the bacterial strain in the composition of the invention is live. For example, the bacterial strain in the composition of the invention is viable. For example, the bacterial strain in the composition of the invention is capable of partially or totally colonising the intestine.

The bacterial strain in the composition of the invention is preferably viable. Preferably it is capable of partially or totally colonising the intestine. The bacterial strain in the composition of the invention may be live and viable. The bacterial strain in the composition of the invention may be live, viable and capable of partially or totally colonising the intestine.

The composition can comprises a mixture of live bacterial strains and bacterial strains that have been killed.

The composition may be administered orally and may be in the form of a tablet, capsule or powder. Encapsulated products are preferred because Fusicatenibacter are obligately anaerobic.

A composition of the invention includes a therapeutically effective amount of a bacterial strain of the invention. A therapeutically effective amount of a bacterial strain is sufficient to exert a beneficial effect upon a patient. A therapeutically effective amount of a bacterial strain may be sufficient to result in delivery to and/or partial or total colonisation of the patient's intestine.

A suitable daily dose of the bacteria, for example for an adult human, may be from about 1×10³ to about 1×10¹¹ colony forming units (CFU); for example, from about 1×10⁷ to about 1×10¹⁰ CFU; in another example from about 1×10⁶ to about 1×10¹⁰ CFU; in another example from about 1×10⁷ to about 1×10¹¹ CFU; in another example from about 1×10⁸ to about 1×10¹⁰ CFU; in another example from about 1×10⁸ to about 1×10¹¹ CFU.

In certain embodiments, the dose of the bacteria is at least 10⁹ cells per day, such as at least 10¹⁰, at least 10¹¹, or at least 10¹² cells per day.

A dose of the composition may comprise the bacterial strain from about 1×10⁶ to about 1×10¹¹ colony forming units (CFU)/g, respect to the weight of the composition. The dose may be suitable for an adult human. For example, the composition may comprise the bacterial strain from about 1×10³ to about 1×10¹¹ CFU/g; for example, from about 1×10⁷ to about 1×10¹⁰ CFU/g; in another example from about 1×10⁶ to about 1×10¹⁰ CFU/g; in another example from about 1×10⁷ to about 1×10¹¹ CFU/g; in another example from about 1×10⁸ to about 1×10¹⁰ CFU/g; in another example from about 1×10⁸ to about 1×10¹¹ CFU/g, from about 1×10⁸ to about 1×10¹⁰ CFU/g. The dose may be, for example, 1 g, 3 g, 5 g, and 10 g.

The composition may be formulated as a probiotic. A probiotic is defined by the FAO/WHO as a live microorganism that, when administered in adequate amounts, confers a health benefit on the host.

Typically, a probiotic is optionally combined with at least one suitable prebiotic compound. In certain embodiments, the probiotic composition of the present invention may include a prebiotic compound in an amount of from about 1 to about 30% by weight, respect to the total weight composition, (e.g. from 5 to 20% by weight. Known prebiotics include commercial products such as inulin and transgalacto-oligosaccharides.

A prebiotic compound is usually a non-digestible carbohydrate such as an oligo- or polysaccharide, or a sugar alcohol, which is not degraded or absorbed in the upper digestive tract.

Potential prebiotics can have a number of beneficial physiological properties including: resistance to digestion in the upper sections of the alimentary tract, can be fermented by the intestinal microbiota, have a beneficial effect on the host's health, can selectively stimulate the growth of the probiotic and is stable in various food or feed processing conditions.

The Carbohydrates may be selected from the group consisting of: fructo-oligosaccharides (or FOS), short-chain fructo-oligosaccharides, inulin, isomalt-oligosaccharides, pectins, xylo-oligosaccharides (or XOS), chitosan-oligosaccharides (or COS), beta-glucans, arable gum modified and resistant starches, polydextrose, D-tagatose, acacia fibers, carob, oats, and citrus fibers. In one aspect, the prebiotics are the short-chain fructo-oligosaccharides (for simplicity shown herein below as FOSs-c.c); said FOSs-c.c. are not digestible carbohydrates, generally obtained by the conversion of the beet sugar and including a saccharose molecule to which three glucose molecules are bonded. The prebiotic can stimulate the growth and activity of bacteria from the genus Fusicatenibacter in the gastrointestinal tract. This allows the bacteria to exert their beneficial effect on the host of treating inflammatory central nervous system diseases or disorders.

Other prebiotic compounds (such as vitamin C, for example), may be included as oxygen scavengers and to improve the delivery and/or partial or total colonisation and survival in vivo. Alternatively, the probiotic composition of the invention may be administered orally as a food or nutritional product, such as milk or whey based fermented dairy product, or as a pharmaceutical product.

The compositions of the invention may be used in combination with another therapeutic compound for treating or preventing central nervous system diseases or disorders. The compositions of the invention may be administered with nutritional supplements that modulate neuroprotection or neuroproliferation. The nutritional supplements comprise or consist of nutritional vitamins. For example, the vitamins can be vitamin B6, magnesium, dimethylglycine (vitamin B16) and vitamin C. The compositions of the invention may be administered in combination with another probiotic.

In certain embodiments, the compositions of the invention are for use in enhancing the effect of a second agent on a central nervous system disease or disorder. The immune modulatory effects of the compositions of the invention may make the brain more susceptible to conventional therapies such as Levodopa, dopamine agonists, MAO-B inhibitors, COMT inhibitors, Glutamate antagonists, or anticholinergics, which are exemplary secondary agents to be administered in combination (sequentially or contemporaneously) with the compositions of the invention.

The compositions of the invention may comprise pharmaceutically acceptable excipients or carriers. Examples of such suitable excipients may be found in the reference [36]. Acceptable carriers or diluents for therapeutic use are well known in the pharmaceutical art and are described, for example, in reference [37]. Examples of suitable carriers include lactose, starch, glucose, methyl cellulose, magnesium stearate, mannitol, sorbitol and the like. Examples of suitable diluents include ethanol, glycerol and water. The choice of pharmaceutical carrier, excipient or diluent can be selected with regard to the intended route of administration and standard pharmaceutical practice. The pharmaceutical compositions may comprise as, or in addition to, the carrier, excipient or diluent any suitable binder(s), lubricant(s), suspending agent(s), coating agent(s), solubilising agent(s). Examples of suitable binders include starch, gelatin, natural sugars such as glucose, anhydrous lactose, free-flow lactose, beta-lactose, corn sweeteners, natural and synthetic gums, such as acacia, tragacanth or sodium alginate, carboxymethyl cellulose and polyethylene glycol. Examples of suitable lubricants include sodium oleate, sodium stearate, magnesium stearate, sodium benzoate, sodium acetate, sodium chloride and the like. Preservatives, stabilizers, dyes and even flavouring agents may be provided in the pharmaceutical composition. Examples of preservatives include sodium benzoate, sorbic acid and esters of p-hydroxybenzoic acid. Antioxidants and suspending agents may be also used.

The compositions of the invention may be formulated as a food product. For example, a food product may provide nutritional benefit in addition to the therapeutic effect of the invention, such as in a nutritional supplement. Similarly, a food product may be formulated to enhance the taste of the composition of the invention or to make the composition more attractive to consume by being more similar to a common food item, rather than to a pharmaceutical composition. In certain embodiments, the composition of the invention is formulated as a milk-based product. The term “milk-based product” means any liquid or semi-solid milk- or whey-based product having a varying fat content. The milk-based product can be, e.g., cow's milk, goat's milk, sheep's milk, skimmed milk, whole milk, milk recombined from powdered milk and whey without any processing, or a processed product, such as yoghurt, curdled milk, curd, sour milk, sour whole milk, butter milk and other sour milk products.

Another important group includes milk beverages, such as whey beverages, fermented milks, condensed milks, infant or baby milks; flavoured milks, ice cream; milk-containing food such as sweets.

The compositions of the invention may comprise one or more bacterial strains of the genus Fusicatenibacter and not contain bacteria from any other genera, or may comprise only de minimis or biologically irrelevant amounts of bacteria from another genera. Thus, the invention provides a composition comprising one or more bacterial strains of the genus Fusicatenibacter, which does not contain bacteria from any other genera or which comprises only de minimis or biologically irrelevant amounts of bacteria from another genera, for use in therapy.

The compositions of the invention may comprise one or more bacterial strains of the species Fusicatenibacter saccharivorans and not contain bacteria from any other species, or may comprise only de minimis or biologically irrelevant amounts of bacteria from another species. Thus the compositions of the invention may comprise one or more bacterial strains of the species Fusicatenibacter saccharivorans and not contain bacteria from any other species, or may comprise only de minimis or biologically irrelevant amounts of bacteria from another species, for use in therapy.

In some embodiments, the composition does not comprise bacteria of the species Clostridium (e.g. C. Difficile) and/or does not comprise bacteria of the species Bacteroides fragilis. In some embodiments the composition does not comprise Bacteroides thetaiotaomicron.

The compositions of the invention may contain a single bacterial species and may not contain any other bacterial species. In certain embodiments, the compositions of the invention contain a single bacterial strain and do not contain any other bacterial strains. For example, the compositions of the invention may comprise a bacterial strain only of the species Fusicatenibacter saccharivorans. Such compositions may comprise only de minimis or biologically irrelevant amounts of other bacterial strains or species. Such compositions may be a culture that is substantially free from other species of organism. In some embodiments, such compositions may be a lyophilisate that is substantially free from other species of organism.

The invention also provides a composition comprising a single bacterial strain of the genus Fusicatenibacter which does not contain bacteria from any other strains or which comprises only de minimis or biologically irrelevant amounts of bacteria from another strain for use in therapy.

The invention also provides a composition comprising a single bacterial strain of the species Fusicatenibacter saccharivorans which does not contain bacteria from any other strains or which comprises only de minimis or biologically irrelevant amounts of bacteria from another strain for use in therapy.

The compositions of the invention may comprise more than one bacterial strain. For example, in some embodiments, the compositions of the invention comprise more than one strain from within the same species (e.g. more than 1, 2, 3, 4, 5, 6, 7, 8, 9, 10, 15, 20, 25, 30, 35, 40 or 45 strains), and, optionally, do not contain bacteria from any other species. In some embodiments, the compositions of the invention comprise fewer than 50 strains from within the same species (e.g. fewer than 45, 40, 35, 30, 25, 20, 19, 18, 17, 16, 15, 14, 13, 12, 11, 10, 9, 8, 7, 6, 5, 4 or 3 strains), and, optionally, do not contain bacteria from any other species. In some embodiments, the compositions of the invention comprise 1-40, 1-30, 1-20, 1-19, 1-18, 1-15, 1-10, 1-9, 1-8, 1-7, 1-6, 1-5, 1-4, 1-3, 1-2, 2-50, 2-40, 2-30, 2-20, 2-15, 2-10, 2-5, 6-30, 6-15, 16-25, or 31-50 strains from within the same species and, optionally, do not contain bacteria from any other species. The invention comprises any combination of the foregoing.

The composition may comprise a microbial consortium. For example, the composition may comprises a Fusicatenibacter strain as part of a microbial consortium. For example, the Fusicatenibacter strain may be present in combination with one or more (e.g. at least 2, 3, 4, 5, 10, 15 or 20) other bacterial strains from the genus Fusicatenibacter and/or other genera with which it can live symbiotically in vivo in the intestine. For example, the composition comprises a bacterial strain of the genus Fusicatenibacter in combination with a bacterial strain from a different genus. In some embodiments, the microbial consortium comprises two or more bacterial strains obtained from a faeces sample of a single organism, e.g. a human. The microbial consortium may not found together in nature. For example, the microbial consortium comprises bacterial strains obtained from faeces samples of at least two different organisms. The microbial consortium can comprises bacteria from two different organisms which are from the same species, e.g. two different humans. For example, the two different organisms are an infant human and an adult human. The microbial consortium can comprise bacteria from two different organisms, which are not from the same species, such as a human and a non-human mammal.

The composition of the invention may additionally comprise a bacterial strain that has the same safety and therapeutic efficacy characteristics as the HT03-11T, KO-38, TT-111 or NCIMB 43527 Fusicatenibacter saccharivorans strains.

In some embodiments in which the composition of the invention comprises more than one bacterial strain, species or genus, the individual bacterial strains, species or genera may be for separate, simultaneous or sequential administration. For example, the composition may comprise all of the more than one bacterial strain, species or genera, or the bacterial strains, species or genera may be stored separately and be administered separately, simultaneously or sequentially. In some embodiments, the more than one bacterial strains, species or genera are stored separately but are mixed together prior to use.

In some embodiments, the bacterial strain for use in the invention is obtained from human adult faeces. In some embodiments in which the composition of the invention comprises more than one bacterial strain, all of the bacterial strains are obtained from human adult faeces or if other bacterial strains are present they are present only in de minimis amounts. The bacteria may have been cultured subsequent to being obtained from the human adult faeces and being used in a composition of the invention.

In some embodiments, the bacterial strain for use in the invention is obtained from human infant faeces. In some embodiments in which the composition of the invention comprises more than one bacterial strain, all of the bacterial strains are obtained from human infant faeces or if other bacterial strains are present they are present only in de minimis amounts. The bacteria may have been cultured subsequent to being obtained from the human infant faeces and being used in a composition of the invention

As mentioned above, in some embodiments, the one or more Fusicatenibacter bacterial strain(s) is/are the only therapeutically active agent(s) in a composition of the invention. In some embodiments, the bacterial strain(s) in the composition is/are the only therapeutically active agent(s) in a composition of the invention.

The compositions for use in accordance with the invention may or may not require marketing approval.

In certain embodiments, the invention provides the above pharmaceutical composition, wherein said bacterial strain is lyophilised. In certain embodiments, the invention provides a lyophilised pharmaceutical composition comprising the bacterial strains as discussed earlier. In certain embodiments, the invention provides the above pharmaceutical composition, wherein said bacterial strain is spray dried. In certain embodiments, the invention provides the above pharmaceutical composition, wherein the bacterial strain is lyophilised or spray dried and wherein it is live. In certain embodiments, the invention provides the above pharmaceutical composition, wherein the bacterial strain is lyophilised or spray dried and wherein it is viable. In certain embodiments, the invention provides the above pharmaceutical composition, wherein the bacterial strain is lyophilised or spray dried and wherein it is capable of partially or totally colonising the intestine. In certain embodiments, the invention provides the above pharmaceutical composition, wherein the bacterial strain is lyophilised or spray dried and wherein it is viable and capable of partially or totally colonising the intestine.

In some cases, the lyophilised bacterial strain is reconstituted prior to administration. In some cases, the reconstitution is by use of a diluent described herein.

The compositions of the invention can comprise pharmaceutically acceptable excipients, diluents or carriers.

In certain embodiments, the invention provides a pharmaceutical composition comprising: a bacterial strain of the invention; and a pharmaceutically acceptable excipient, carrier or diluent; wherein the bacterial strain is in an amount sufficient to treat an inflammatory neurodegenerative disorder when administered to a subject in need thereof.

In certain embodiments, the invention provides pharmaceutical composition comprising: a bacterial strain of the invention; and a pharmaceutically acceptable excipient, carrier or diluent; wherein the bacterial strain is in an amount sufficient to treat or prevent an inflammatory neurodegenerative disorder.

In certain embodiments, the invention provides the above pharmaceutical composition, wherein the amount of the bacterial strain is from about 1×10³ to about 1×10¹¹ colony forming units per gram with respect to a weight of the composition.

In certain embodiments, the invention provides the above pharmaceutical composition, wherein the composition is administered at a dose of 1 g, 3 g, 5 g or 10 g.

In certain embodiments, the invention provides the above pharmaceutical composition, wherein the composition is administered by a method selected from the group consisting of oral, rectal, subcutaneous, nasal, buccal, and sublingual.

In certain embodiments, the invention provides the above pharmaceutical composition, comprising a carrier selected from the group consisting of lactose, starch, glucose, methyl cellulose, magnesium stearate, mannitol and sorbitol.

In certain embodiments, the invention provides the above pharmaceutical composition, comprising a diluent selected from the group consisting of ethanol, glycerol and water.

In certain embodiments, the invention provides the above pharmaceutical composition, comprising an excipient selected from the group consisting of starch, gelatin, glucose, anhydrous lactose, free-flow lactose, beta-lactose, corn sweetener, acacia, tragacanth, sodium alginate, carboxymethyl cellulose, polyethylene glycol, sodium oleate, sodium stearate, magnesium stearate, sodium benzoate, sodium acetate and sodium chloride.

In certain embodiments, the invention provides the above pharmaceutical composition, further comprising at least one of a preservative, an antioxidant and a stabilizer.

In certain embodiments, the invention provides the above pharmaceutical composition, comprising a preservative selected from the group consisting of sodium benzoate, sorbic acid and esters of p-hydroxybenzoic acid.

In certain embodiments, the invention provides the above pharmaceutical composition, wherein said bacterial strain is lyophilised.

In certain embodiments, the invention provides the above pharmaceutical composition, wherein when the composition is stored in a sealed container at about 4° C. or about 25° C. and the container is placed in an atmosphere having 50% relative humidity, at least 80% of the bacterial strain as measured in colony forming units, remains after a period of at least about: 1 month, 3 months, 6 months, 1 year, 1.5 years, 2 years, 2.5 years or 3 years.

In some embodiments, the composition of the invention is provided in a sealed container comprising a composition as described herein. In some embodiments, the sealed container is a sachet or bottle. In some embodiments, the composition of the invention is provided in a syringe comprising a composition as described herein.

The composition of the present invention may, in some embodiments, be provided as a pharmaceutical formulation. For example, the composition may be provided as a tablet or capsule. In some embodiments, the capsule is a gelatine capsule (“gel-cap”). The capsule can be a hard or a soft capsule. In some embodiments, the formulation is a soft capsule. Soft capsules are capsules which may, owing to additions of softeners, such as, for example, glycerol, sorbitol, maltitol and polyethylene glycols, present in the capsule shell, have a certain elasticity and softness. Soft capsules can be produced, for example, on the basis of gelatine or starch. Gelatine-based soft capsules are commercially available from various suppliers. Depending on the method of administration, such as, for example, orally or rectally, soft capsules can have various shapes, they can be, for example, round, oval, oblong or torpedo-shaped. Soft capsules can be produced by conventional processes, such as, for example, by the Scherer process, the Accogel process or the droplet or blowing process.

In some embodiments, the compositions of the invention are administered orally. Oral administration may involve swallowing, so that the compound enters the gastrointestinal tract.

Pharmaceutical formulations suitable for oral administration include solid plugs, solid microparticulates, semi-solid and liquid (including multiple phases or dispersed systems) such as tablets; soft or hard capsules containing multi- or nano-particulates, liquids (e.g. aqueous solutions), emulsions or powders; lozenges (including liquid-filled); chews; gels; fast dispersing dosage forms; films; ovules; sprays; and buccal/mucoadhesive patches.

In some embodiments the pharmaceutical formulation is an enteric formulation, i.e. a gastro-resistant formulation (for example, resistant to gastric pH) that is suitable for delivery of the composition of the invention to the intestine by oral administration. Enteric formulations may be particularly useful when the bacteria or another component of the composition is acid-sensitive, e.g. prone to degradation under gastric conditions.

In some embodiments, the enteric formulation comprises an enteric coating. In some embodiments, the formulation is an enteric-coated dosage form. For example, the formulation may be an enteric-coated tablet or an enteric-coated capsule, or the like. The enteric coating may be a conventional enteric coating, for example, a conventional coating for a tablet, capsule, or the like for oral delivery. The formulation may comprise a film coating, for example, a thin film layer of an enteric polymer, e.g. an acid-insoluble polymer.

In some embodiments, the enteric formulation is intrinsically enteric, for example, gastro-resistant without the need for an enteric coating. Thus, in some embodiments, the formulation is an enteric formulation that does not comprise an enteric coating. In some embodiments, the formulation is a capsule made from a thermogelling material. In some embodiments, the thermogelling material is a cellulosic material, such as methylcellulose, hydroxymethylcellulose or hydroxypropylmethylcellulose (HPMC). In some embodiments, the capsule comprises a shell that does not contain any film forming polymer. In some embodiments, the capsule comprises a shell and the shell comprises hydroxypropylmethylcellulose and does not comprise any film forming polymer (e.g. see [38]). In some embodiments, the formulation is an intrinsically enteric capsule (for example, Vcaps™ from Capsugel).

Culturing Methods

The bacterial strains for use in the present invention can be cultured using standard microbiology techniques as detailed in, for example, references [39-41].

The solid or liquid medium used for culture may for example be YCFA agar or YCFA medium. YCFA medium may include (per 100 ml, approximate values): Casitone (1.0 g), yeast extract (0.25 g), NaHCO₃ (0.4 g), cysteine (0.1 g), K₂HPO₄ (0.045 g), KH₂PO₄ (0.045 g), NaCl (0.09 g), (NH₄)₂SO₄ (0.09 g), MgSO₄.7H₂O (0.009 g), CaCl₂) (0.009 g), resazurin (0.1 mg), hemin (1 mg), biotin (1 μg), cobalamin (1 μg), p-aminobenzoic acid (3 μg), folic acid (5 μg), and pyridoxamine (15 μg).

General

The practice of the present invention will employ, unless otherwise indicated, conventional methods of chemistry, biochemistry, molecular biology, immunology and pharmacology, within the skill of the art. Such techniques are explained fully in the literature. See, e.g., references [42-49], etc.

The term “comprising” encompasses “including” as well as “consisting” e.g. a composition “comprising” X may consist exclusively of X or may include something additional e.g. X+Y.

The term “about” in relation to a numerical value x is optional and means, for example, x±10%.

The word “substantially” does not exclude “completely” e.g. a composition which is “substantially free” from Y may be completely free from Y. Where necessary, the word “substantially” may be omitted from the definition of the invention.

A “disease” results from a pathophysiological response to external or internal factors. A “disorder” is a disruption of the disease to the normal or regular functions in the body or a part of the body. A “condition” is an abnormal state of health that interferes with the usual activities or feeling of wellbeing.

References to a percentage sequence identity between two nucleotide sequences means that, when aligned, that percentage of nucleotides are the same in comparing the two sequences. References to a percentage sequence similarity (also known a sequence homology) between two nucleotide sequences measures the degree of resemblance between two sequences and demonstrates the extent to which different nucleotide bases are aligned. This alignment and the percent homology or sequence identity can be determined using software programs known in the art, for example those described in section 7.7.18 of ref. [50]. A preferred alignment is determined by the Smith-Waterman homology search algorithm using an affine gap search with a gap open penalty of 5 or 12, most preferably 5, and a gap extension penalty of 2, BLOSUM matrix of 62. The Smith-Waterman homology search algorithm is disclosed in ref. [51].

Unless specifically stated, a process or method comprising numerous steps may comprise additional steps at the beginning or end of the method, or may comprise additional intervening steps. Also, steps may be combined, omitted or performed in an alternative order, if appropriate.

Various embodiments of the invention are described herein. It will be appreciated that the features specified in each embodiment may be combined with other specified features, to provide further embodiments. In particular, embodiments highlighted herein as being suitable, typical or preferred may be combined with each other (except when they are mutually exclusive).

All patent and literature references cited in the present specification are hereby incorporated by reference in their entirety.

Any reference to a method for treatment comprising administering an agent to a patient, also covers that agent for use in said method for treatment, as well as the use of the agent in said method for treatment, and the use of the agent in the manufacture of a medicament.

The following examples are offered for illustrative purposes only, and are not intended to limit the scope of the present invention in any way.

### MODES FOR CARRYING OUT THE INVENTION

### Example 1—Effect of Fusicatenibacter saccharivorans on Intestinal Permeability

Summary

This study investigated the effect of a strain identified as belonging to the Fusicatenibacter saccharivorans species by 16S homology and MALDI-TOF analysis on intestinal permeability of mouse ileum and colon samples. Excessive permeability, or ‘leakiness’, of the intestine is associated with a number of inflammatory disorders of the gut.

Materials and Methods

Male BALB/c mice received oral gavage (200 μL volume) of 1×10⁹ CFU Fusicatenibacter saccharivorans for 6 consecutive days. On day 7, the animals were euthanized by cervical dislocation, and the distal ileum and colon were removed, placed in chilled Krebs solution opened along the mesenteric line and carefully rinsed. Preparations were then placed in Ussing chambers (Harvard Apparatus, Kent, UK, exposed area of 0.12 cm²) as described previously (Hyland and Cox, 2005) with oxygenated (95% O2, 5% CO₂) Krebs buffer maintained at 37° C. 4 kDa FITC-dextran was added to the mucosal chamber at a final concentration of 2.5 mg/mL; 200 μL samples were collected from the serosal chamber every 30 min for the following 3 h and fluorescence in those samples measured.

Results

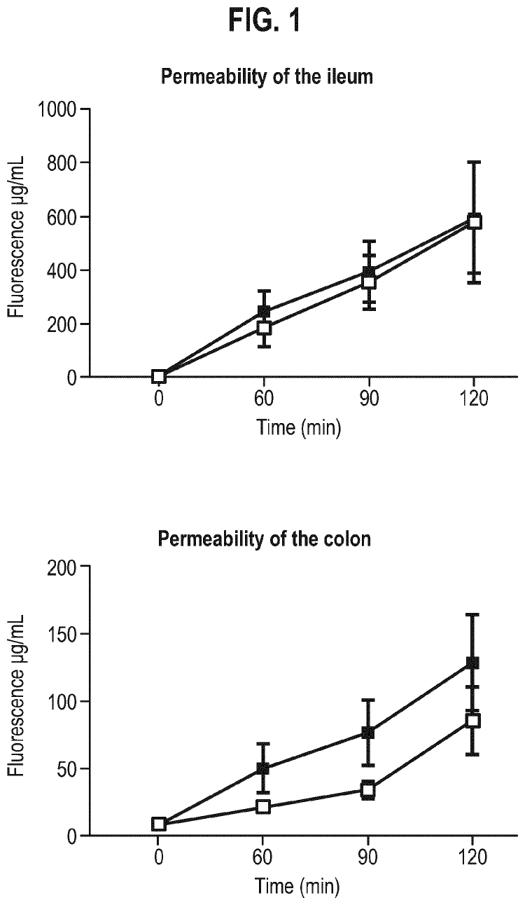

FIG. 1 shows that treatment with Fusicatenibacter saccharivorans decreased permeability of the colon (FIG. 1B). Therefore, bacterial strains of the invention may be useful in the improvement of gut barrier function and thus the treatment or prevention of diseases associated with intestinal permeability.

### Example 2—Effect of Fusicatenibacter saccharivorans on the Kynurenine Pathway

Summary

This study investigated the effect of Fusicatenibacter saccharivorans on enzymes in the kynurenine pathway and tryptophan catabolism. Indoleamine 2,3 dioxygenase-1 (IDO1) is an enzyme that initiates tryptophan catabolism along a pathway that generates several bioactive kynurenine-based metabolites. Tryptophan hydroxylase-1 (TPH-1) catalyze the formation of 5-hydroxy-L-tryptophan (5-HTP) from L-tryptophan, the first and rate-limiting step in the biosynthesis of 5-HT, which is an intermediate in the serotonin pathway.

Materials and Methods

Method

Male BALB/c mice received oral gavage (200 μL volume) of 1×10⁹ CFU of the relevant bacterial strain for 6 consecutive days. On day 7, the animals were euthanized. Intestinal tissue (1 cm segments of ileum and colon) were excised. Total RNA was extracted using the mirVana™ miRNA Isolation kit (Ambion/Llife technologies, Paisley, UK) and DNase treated (Turbo DNA-free, Ambion/life technologies) according to the manufacturers recommendations. RNA was quantified using NanoDrop™ spectrophotometer (Thermo Fisher Scientific Inc., Wilmington, Del., USA) according to the manufacturer's instructions. RNA quality was assessed using the Agilent Bioanalyzer (Agilent, Stockport, UK) according to the manufacturer's procedure and an RNA integrity number (RIN) was calculated. RNA with RIN value >7 was used for subsequent experiments. RNA was reverse transcribed to cDNA using the Applied Biosystems High Capacity cDNA kit (Applied Biosystems, Warrington, UK) according to manufacturer's instructions. Briefly, Multiscribe Reverse Transcriptase (50 U/L) was added as part of RT master mix, incubated for 25° C. for 10 min, 37° C. for 2 h, 85° C. for 5 min and stored at 4° C. Quantitative PCR was carried out using probes (6 carboxy fluorescein—FAM) designed by Applied Biosystems to mouse specific targeted genes, while using β-actin as an endogenous control. Amplification reactions contained 1 μl cDNA, 5 μl of the 2×PCR Master mix (Roche), 900 nM of each primer and were brought to a total of 10 W by the addition of RNase-free water. All reactions were performed in triplicate using 96-well plates on the LightCycler®480 System. Thermal cycling conditions were as recommended by the manufacturer (Roche) for 55 cycles. To check for amplicon contamination, each run contained no template controls in triplicate for each probe used. Cycle threshold (Ct) values were recorded. Data was normalized using β-actin and transformed using the 2-ΔΔCT method and presented as a fold change vs. control group.

Results

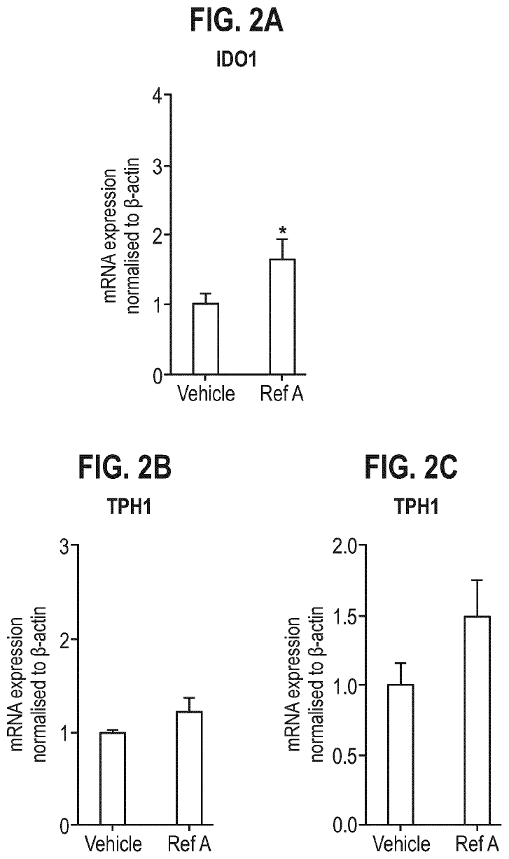

IDO1 expression in the colon was significantly higher in mice that had received Fusicatenibacter saccharivorans compared to the untreated controls (FIG. 2A). There was also a trend towards increased expression of TPH1 in the ileum (FIG. 2B) and colon (FIG. 2C). The data show that bacteria from the genus Fusicatenibacter can modulate the serotonin and kynurenine pathways.

### Example 3—Effect of Fusicatenibacter saccharivorans on Short-Chain Fatty Acid Production

Summary

This study investigated the effect of Fusicatenibacter saccharivorans on the production of short-chain fatty acids (SCFAs) in mice. SCFAs, which include acetate, propionate, valerate, butyrate, isobutyrate and isovalerate are microbial byproducts of dietary fibre. An increase in any SCFA suggests an increase in productivity of the microbiota and is a desirable trait.

Materials and Methods

Male BALB/c mice received oral gavage (200 μL volume) of 1×10⁹ CFU of the relevant bacterial strain for 6 consecutive days. On day 7, the animals were euthanized. The caecum was removed, weighed and stored at −80° C. for SCFAs analysis. Caecum content was mixed and vortexed with MilliQ water and incubated at room temperature for 10 min. Supernatants were obtained by centrifugation (10000 g, 5 min, 4° C.) to pellet bacteria and other solids and filtration by 0.2 m. It was transferred to a clear GC vial and 2-Ethylbutyric acid (Sigma) was used as the internal standard. The concentration of SCFA was analyzed using a Varian 3500 GC flame-ionization system, fitted with a with a ZB-FFAP column (30 m×0.32 mm×0.25 mm; Phenomenex). A standard curve was built with different concentrations of a standard mix containing acetate, propionate, iso-butyrate, n-butyrate, isovalerate and valerate (Sigma). Peaks were integrated by using the Varian Star Chromatography Workstation version 6.0 software. All SCFA data are expressed as mol/g.

Results

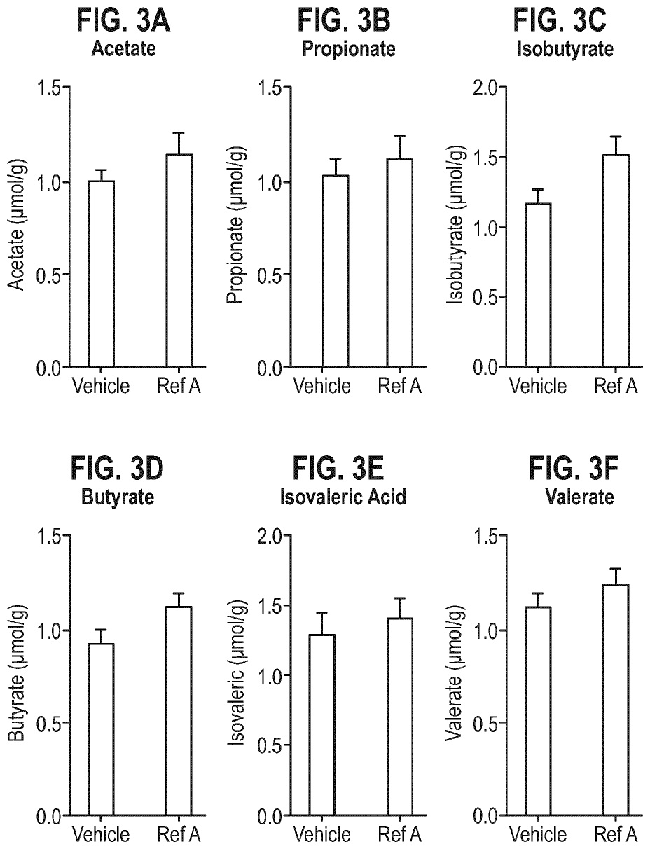

FIG. 3 shows that treatment with Fusicatenibacter saccharivorans resulted in a general increase in the production of acetate (A), propionate (B), isobutyrate (C), butyrate (D), isovalerate (E) and valerate (F). SCFAs produced by bacteria in the microbiome are key mediators of the beneficial effects elicited by the gut microbiome. These data suggest that bacteria from the genus Fusicatenibacter may be useful in promoting productivity of the microbiota, and therefore useful in the treatment or prevention of diseases associated with a reduced productivity of the microbiota. SCFA can regulate the immune response, therefore these data suggest that bacteria from the genus Fusicatenibacter may be useful in the treatment of inflammatory conditions.

The inventors have demonstrated for the first time that bacteria from the genus Fusicatenibacter are can produce butyrate. Sodium butyrate is known to attenuate social behaviour deficits in an autism mouse model, therefore the compositions of the invention may be particularly effective at treating or preventing an autism spectrum disorder.

### Example 4—Effect of Fusicatenibacter saccharivorans on Gene Expression in the Brain

Summary

This study investigated the effect of Fusicatenibacter saccharivorans on the expression of certain genes of interest in the brain. mRNA levels for markers for the oxytocinergic system (oxytocin receptor and vasopressin receptor), endocrine system (mineralocorticoid (Nr3c1); glucocorticoid receptor (Nr3c2); corticosterone releasing factor (CRF) and receptors; Brain derived neurotrophic factor (BDNF)), immune system (II-6, TNF-α, TLR-4); and neurotransmitter systems (NMDA receptor 2A (Grin2A); NMDA receptor 2B (Grin2B); GABAA receptor subunit A2; GABAB receptor subunit B1; serotonin 2C) were assessed in the amygdala, hippocampus and prefrontal cortex (PFC), which are key brain regions of the limbic system involved in emotional response.

Materials and Methods

Method

Male BALB/c mice received oral gavage (200 μL volume) of 1×10⁹ CFU of Fusicatenibacter saccharivorans for 6 consecutive days. On day 7, the animals were euthanized. The brain was quickly excised, dissected and each brain region was snap-frozen on dry ice and stored at −80° C. for further analysis. Total RNA was extracted using the mirVana™ miRNA Isolation kit (Ambion/Llife technologies, Paisley, UK) and DNase treated (Turbo DNA-free, Ambion/life technologies) according to the manufacturers recommendations. RNA was quantified using NanoDrop™ spectrophotometer (Thermo Fisher Scientific Inc., Wilmington, Del., USA) according to the manufacturer's instructions. RNA quality was assessed using the Agilent Bioanalyzer (Agilent, Stockport, UK) according to the manufacturer's procedure and an RNA integrity number (RIN) was calculated. RNA with RIN value >7 was used for subsequent experiments. RNA was reverse transcribed to cDNA using the Applied Biosystems High Capacity cDNA kit (Applied Biosystems, Warrington, UK) according to manufacturer's instructions. Briefly, Multiscribe Reverse Transcriptase (50 U/L) was added as part of RT master mix, incubated for 25° C. for 10 min, 37° C. for 2 h, 85° C. for 5 min and stored at 4° C. Quantitative PCR was carried out using probes (6 carboxy fluorescein—FAM) designed by Applied Biosystems to mouse specific targeted genes, while using β-actin as an endogenous control. Amplification reactions contained 1 μl cDNA, 5 μl of the 2×PCR Master mix (Roche), 900 nM of each primer and were brought to a total of 10 μl by the addition of RNase-free water. All reactions were performed in triplicate using 96-well plates on the LightCycler®480 System. Thermal cycling conditions were as recommended by the manufacturer (Roche) for 55 cycles. To check for amplicon contamination, each run contained no template controls in triplicate for each probe used. Cycle threshold (Ct) values were recorded. Data was normalized using β-actin and transformed using the 2-ΔΔCT method and presented as a fold change vs. control group.

Results

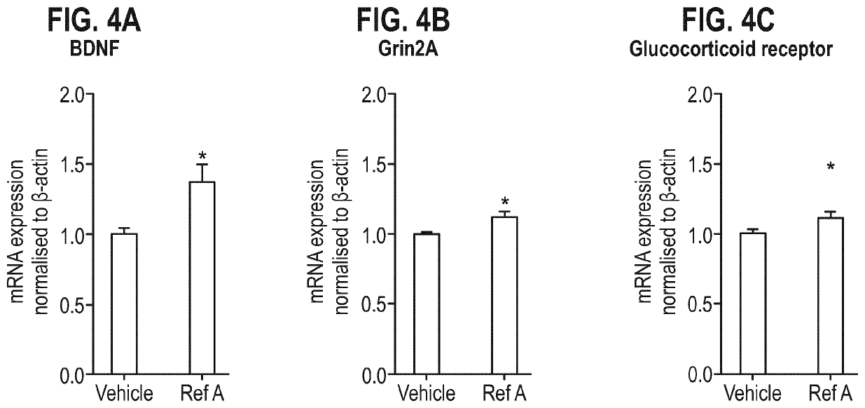

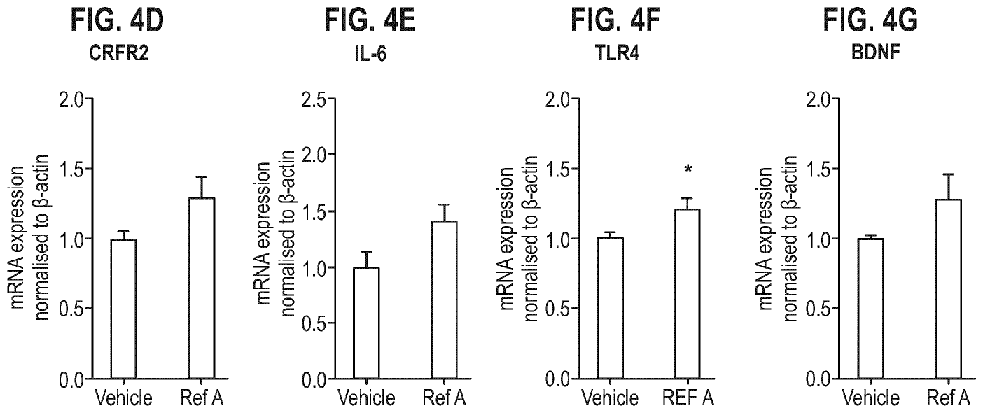

As shown in FIG. 4, from the genes tested, a significant increase in the expression of BDNF (A), Grin2a (B) and glucocorticoid receptor (C) and a trend for increased CRFR2 (D) and IL-6 (E) expression in the amygdala, and a significant increase in TLR-4 (F) expression and trend for an increased expression of BDNF (G) in the PFC was observed in mice which have been treated with Fusicatenibacter saccharivorans, relative to treatment with vehicle control. This indicates that bacteria from the genus Fusicatenibacter are capable of modulating the levels of expression of proteins in the brain, therefore they may be useful in the treatment or prevention of CNS diseases, disorders or conditions, such as autism.

BDNF is a neurotrophic growth factor that has been shown to enhance neuron differentiation and survival. Therefore, bacteria from the genus Fusicatenibacter may be particularly useful in a method of enhancing nerve cell survival in the treatment or prevention of CNS diseases, disorders or conditions.

Glucocorticoids have anti-inflammatory, anti-proliferative, pro-apoptotic and anti-angiogenic roles. Therefore, the ability of bacteria from the genus Fusicatenibacter to increase glucocorticoid receptor expression in the brain indicates that bacteria from the genus Fusicatenibacter may be useful in therapy, and particularly useful in treating or preventing CNS diseases, disorders or conditions.

### Example 5—Effect of Fusicatenibacter saccharivorans on Behaviour in Models of Autism Spectrum Disorders

Summary

This study investigated the effect of Fusicatenibacter saccharivorans on a number of behavioural readouts including stereotype behaviour (marble burying), social behaviour (social transfer of food preference, female urine sniffing test, 3-chamber social interaction test) and depression/acute stress (forced swim test) in both an environmental animal model (maternal immune activation model) and a genetic animal model (BTBR mouse strain) of autism spectrum disorder.

The BTBR mouse was chosen as it represents an idiopathic model of autism with an underlying genetic component. These mice display profound autistic-like behaviour, with deficits in sociability and social cognition, heightened anxiety and stereotyped behaviours such as increased grooming. In addition to the behavioural profile, BTBR mice exhibit increased permeability of both the large and small intestine, prolonged intestinal motility and reduced enteric serotonin production which collectively suggests dysfunction of the gastrointestinal system [52]. The BTBR mouse model uses inbred, genetically modified mice that display a robust autistic-like phenotype. Deficits in social behaviours, increased repetitive behaviours and increased anxiety-related behaviours have been reported in this strain [53]. Due to this robust behavioural phenotype, the BTBR mouse is an ideal animal model to assess the efficacy of novel therapeutic agents for the treatment of autistic-related behaviours. Alleviation of such symptoms by a live bacterial strain can also be indicative of efficacy of the bacterial strain in the treatment of other psychiatric or neurological diseases.

The MIA mouse model uses an environmental immune challenge in pregnant mice in order to trigger the core symptoms of autism spectrum disorder in the offspring. MIA mice typically display stereotyped behaviour (as shown by the grooming and marble burying tests) and deficits in social communication (as shown by the social play, 3-chamber social interaction, and social transmission of food preference tests). The offspring display the three core symptoms of autism (reduced communication; reduced sociability; and increased repetitive or stereotyped behaviour) and therefore provide a suitable model in which to determine whether administration of a therapeutic can alleviate the behavioural phenotypes associated with autistic spectrum disorders and indeed in a number of other neurological disorders. It is well established that alteration of behavioural phenotypes in animal models is indicative of a potentially clinically relevant intervention, irrespective of an understanding of the underlying biological or physiological mechanism [54].

Collectively, the BTBR and MIA represent appropriate models of dysregulation of the microbiota-gut-brain axis, which may contribute towards their autistic-like phenotype.

Materials and Methods

BTBR Mouse Model

BTBR animals were bred in house with brother-sister mating. The male offspring from these animals were separated from their mothers at 3 weeks old and daily administration of Fusicatenibacter saccharivorans or control commenced at 8 weeks of age. Behaviour started at 11 weeks old. A control age-matched C57/B16 group was included as a reference control group.

MIA Mouse Model

Female C57/B16 mice (8 weeks old) and age matched males were purchased from Harlan UK. After 1-week habituation these animals were mated. At embryonic day 12.5, females received either an injection of the viral mimetic poly-IC to activate the maternal immune system, or a saline vehicle injection. The male offspring from these animals were separated from their mothers at 3 weeks old and daily administration of the live biotherapeutic or control commenced at 8 weeks of age. Behaviour started at 11 weeks old.

Marble Burying

Mice were individually placed in a novel plexiglass cage (35×28×18.5 cm, L×W×H), filled up with sawdust (5-10 cm) and 20 marbles on top of it (five rows or marbles regularly spaced 2 cm away from the walls and 2 cm apart). Thirty minutes later, the number of marbles buried for more than ⅔ of their surface was counted. A higher number of marbles buried represents a heightened state of stereotype behaviour or higher levels of anxiety (neophobia).

Social Behaviour

The social testing apparatus was a rectangle, three-chambered box. Each chamber was 20 cm L×40 cm W×22 cm H. Dividing walls were made with small circular openings (5 cm in diameter) allowing access into each chamber. Two identical wire cup-like cages, with a bottom diameter of 10 cm, 13 cm in height and bars spaced 1.2 cm, allowing nose contact between the bars, but prevented fighting, were placed inside each side chamber in bilaterally symmetric positions. The test has three phases of 10 min each: 1) habitation 2) mouse versus object 3) novel mouse versus familiar mouse. Experiments were videotaped using a ceiling camera for further parameters analysis using Ethovision software (3.1 version, Noldus, TrackSys, Nottingham, UK). For the first phase the test mouse was placed into the middle chamber and allowed to explore the entire box with empty small wire cages inside for a 10-min habituation session. After the habituation period, the test mouse is removed from the testing box for a short interval while an object is placed in one side chamber and an unfamiliar conspecific male mouse (no prior contact with the test subject) in the other side chamber, both enclosed in a wire cup-like cage. During phase two, the test mouse is placed in the middle chamber and allowed to explore the entire box for 10 min. The amount of time spent exploring the object or mouse in each chamber and the number of entries into each chamber were evaluated. The location of the unfamiliar mouse in the left vs right side chamber was systematically alternated between trials. An entry was defined as all four paws in one chamber. During the third phase an object was replaced with an unfamiliar mouse serving as a novel mouse and in the other chamber the mouse used in phase two was kept the same, now serving as familiar mouse. After every trial, all chambers and cup-like wire cages were cleaned with 10% ethanol, dried and ventilated for a few minutes to prevent olfactory cue bias and to ensure proper disinfection. Lack of innate side preference was confirmed during the initial 10 min of habituation to the entire arena. Control animals naturally are more interested in a conspecific mouse more than an inanimate object (sociability). In a similar vein, control animals spend more time interacting with a novel unfamiliar mouse then one they have already had interactions with. Some animal models, as used here, have deficits in this social paradigm.

Forced Swim

Mice were individually placed in a clear glass cylinder (24×21 cm diameter), containing 15-cm-depth water (25±0.5° C.). Water was changed between each animal. The test lasted 6 min and experiments were videotaped using a numeric tripod-fixed camera; data were further scored twice using the videos (Video Media Player software) and averaged by an experimenter blind to conditions. The latency to immobility was scored. The time of immobility (s) was measured for the last 4 min of the test, with immobility being defined as a total absence of movement except slight motions to maintain the head above the water. An increase in immobility means an increase in depressive-like behaviour as the animal has resigned itself to its situation (learned helplessness).

Method

Animals received daily oral gavage of PBS or Fusicatenibacter saccharivorans prepared to 10⁹ cfu/mL in PBS. Dosing continued daily throughout the behavioural paradigm.

Results

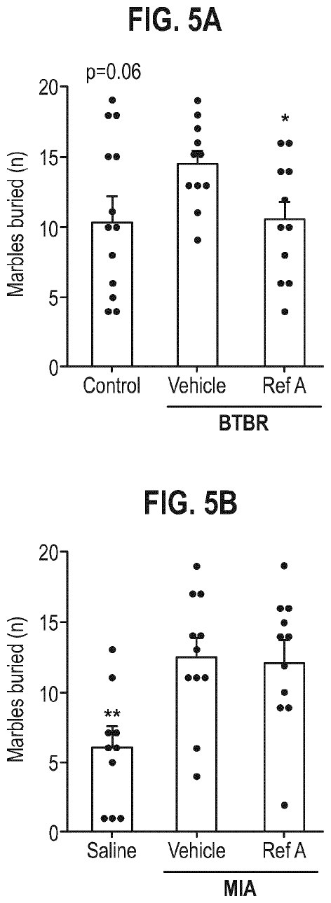

As shown in FIG. 5A, BTBR mice treated with Fusicatenibacter saccharivorans exhibited significantly reduced stereotype-related behaviour compared to the vehicle control. Treatment with the bacteria resulted in stereotype-related behaviour (i.e. the number of marbles buried) that was the comparable to control mice which had no disease symptoms.

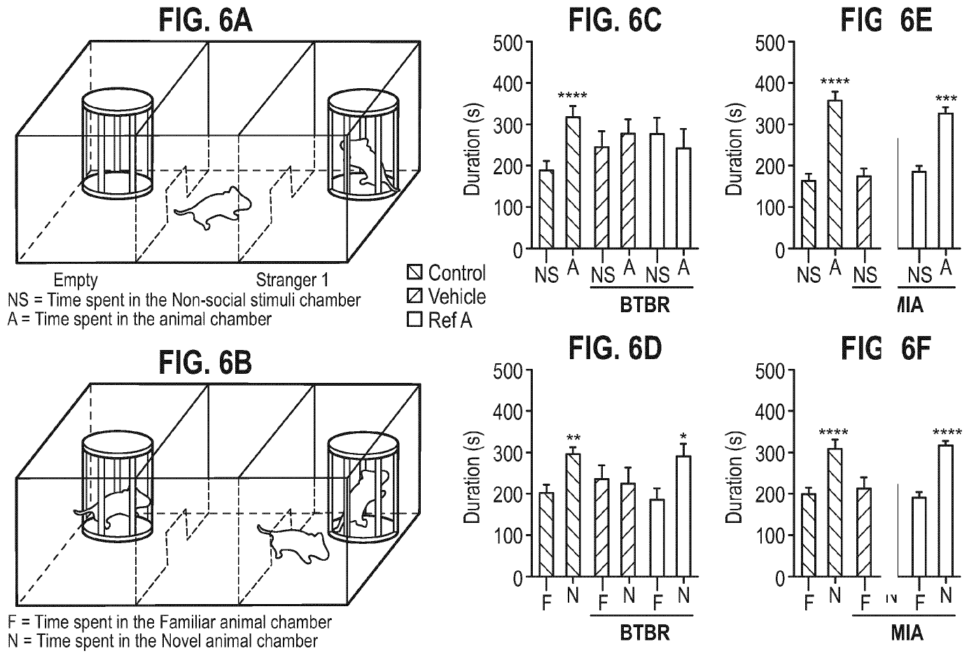

FIG. 6 shows that treatment with Fusicatenibacter saccharivorans resulted in more social behaviour in the MIA model, as MIA mice spent significantly more time in the animal chamber than the non-social stimuli chamber (FIG. 6 E). In both the BTBR and MIA models mice spent significantly more time in the novel animal chamber than the familiar animal chamber (FIG. 6 D and F). These data show that the social behaviour exhibited after treatment with Fusicatenibacter saccharivorans is comparable with non-diseases control mice.

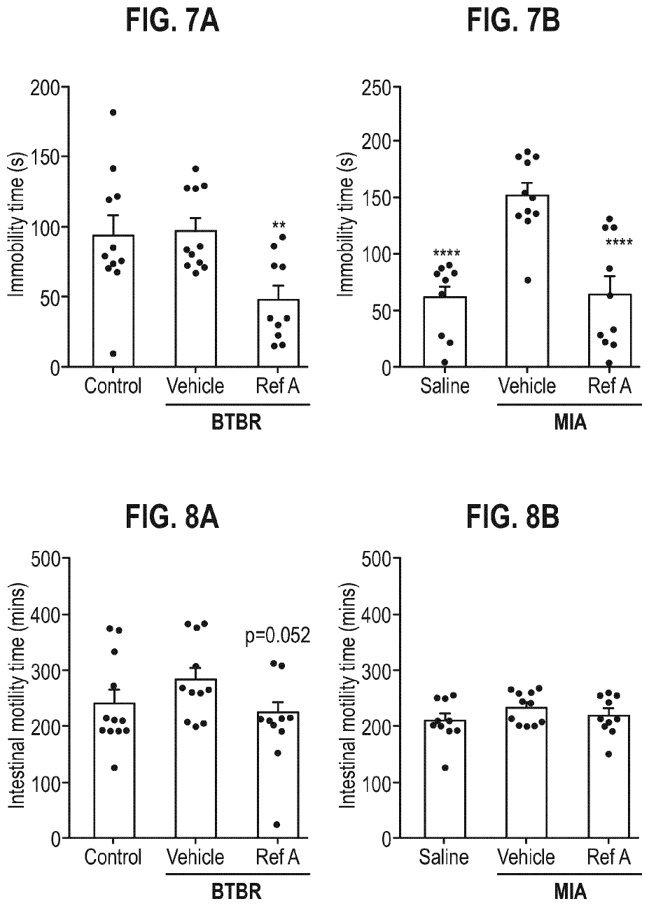

Treatment with Fusicatenibacter saccharivorans also had a positive effect on depressive-like behaviour. Both BTBR and MIA mice exhibited significantly less immobility time in the forced swim test, as shown in FIG. 7.

In conclusion, Fusicatenibacter saccharivorans was shown to be effective in the treatment of stereotyped, social behaviour and depressive-like behaviours in both the BTBR and MIA mouse model. Therapies that reverse behavioural and biological phenotypes in mouse models of autism are expected to be effective against human disease.

The EMA Guidelines on the clinical development of medicinal products for the treatment of autism spectrum disorder state that, due to the heterogeneity of the diseases, it may not be possible to achieve a significant effect on all core symptoms with a single compound, and so short term efficacy has to be demonstrated on at least one core symptom. Fusicatenibacter saccharivorans has shown effective treatment of at least one core symptom of autistic spectrum disorder, therefore it can be expected to be effective against human disease. Similarly, other central nervous system disorders or conditions display complex pathology with multiple different symptoms, and have few approved treatments. Therefore, it is understood that an effective treatment does not need to treat all symptoms of a central nervous system disease or disorder. A treatment would be considered therapeutically useful if it treated one of the symptoms associated with a central nervous system disease or disorder.

In conclusion, these results indicate that bacteria from the genus Fusicatenibacter may be useful in the treatment or prevention of autism spectrum disorders.

### Example 6—Effect of Fusicatenibacter saccharivorans on Intestinal Motility and Gastrointestinal Permeability in Models of Autism Spectrum Disorders

Summary

This study investigated the effect of Fusicatenibacter saccharivorans on gut mobility and permeability in both MIA and BTBR autism spectrum models.

Materials and Methods

Mouse Models

The BTBR and MIA models were generated and maintained as described in Example 6.

Method for Assessing Intestinal Motility

This procedure involves the oral administration of a given amount of a non-toxic, coloured marker (Carmin Red) to determine motility of the gut. Time to excretion of the first coloured faecal bolus is recorded as ‘time of whole gut transit’ and is used as an index of peristaltic motility in the whole intestine. Mice are single housed for 3h prior to the assay to allow habituation to a new cage. Carmin red dye (100-200 μl of 6% Carmin red in 0.5% methylcellulose per mouse) is given orally by gavage. Each cage is visually inspected every 10 min. The time of the first coloured bolus (red) is recorded.

Following the appearance of the first coloured bolus mice are returned to normal housing conditions.

Method for Assessing Gastrointestinal Permeability

The method was performed as described in Example 1.

Results

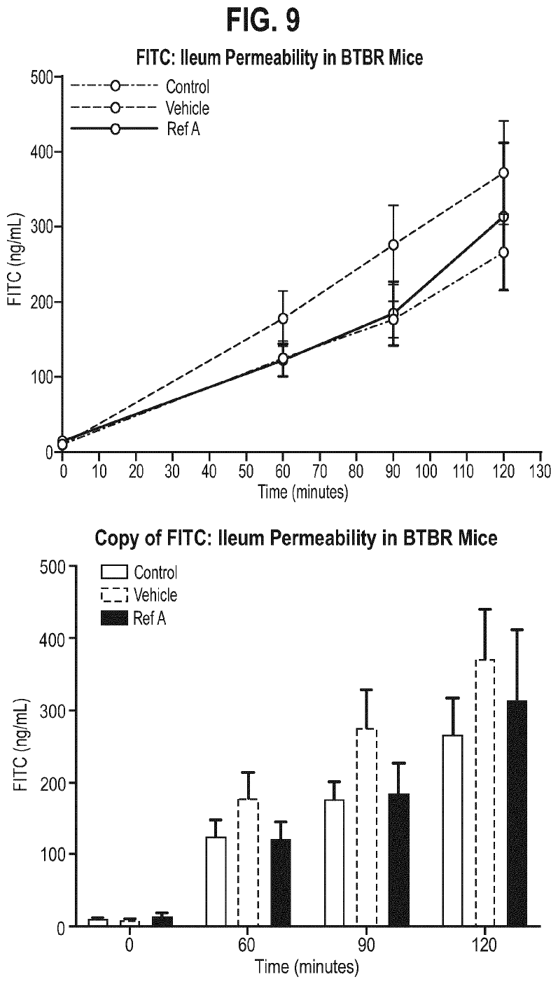

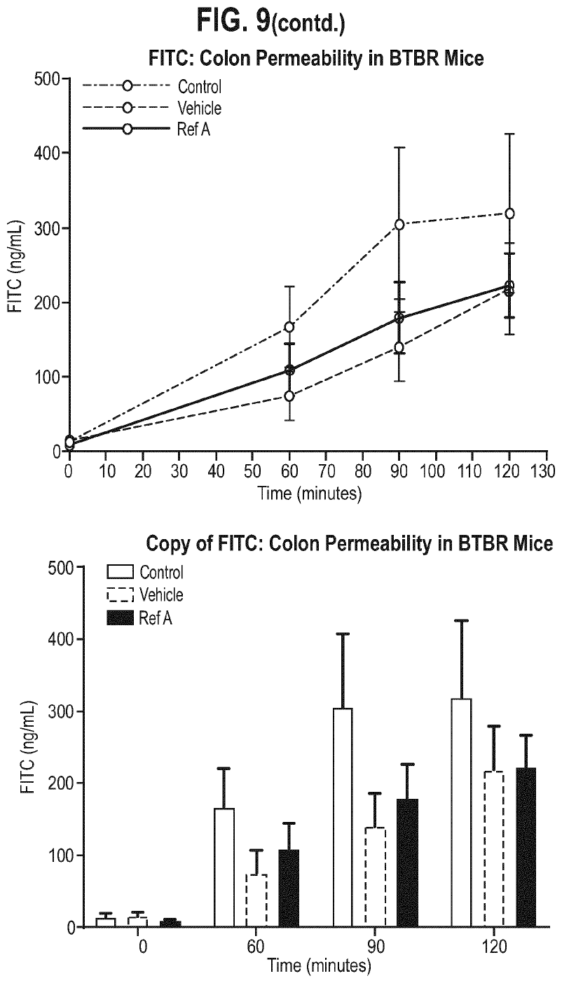

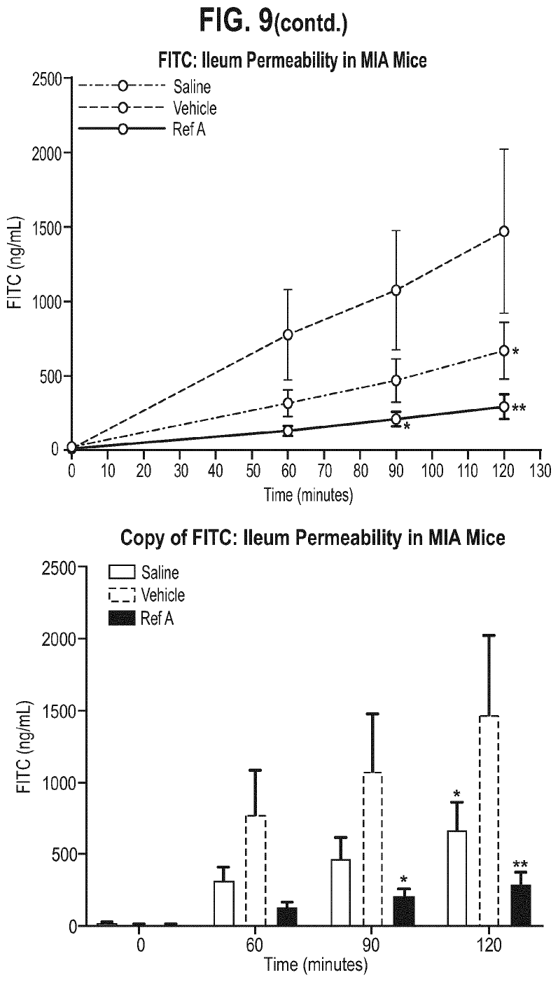

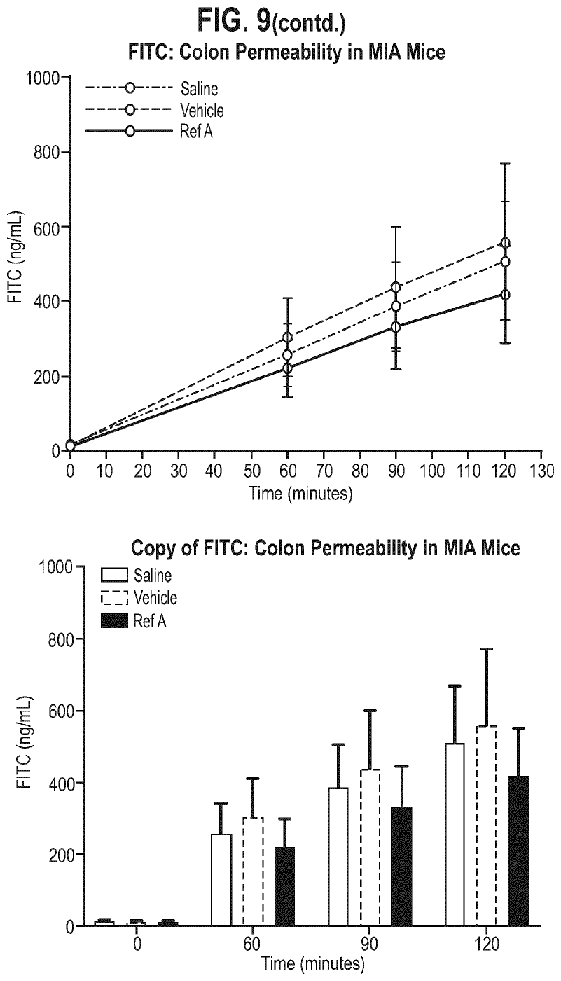

As shown in FIG. 8, Fusicatenibacter saccharivorans was able to reduce intestinal motility in both the BTBR and MIA models. Treatment with Fusicatenibacter saccharivorans also lead to a reduction in permeability in the ileum and the colon (FIG. 9). These results demonstrate that Fusicatenibacter saccharivorans can reverse the prolonged intestinal motility and increased gastrointestinal permeability observed in the MIA model, which relates to the dysfunction of the gastrointestinal system. Therefore, without wishing to be bound by any particular theory, Fusicatenibacter saccharivorans may be particularly effective at treating autism spectrum disorders by correcting the dysfunction of the gastrointestinal system.

The data suggest that bacteria from the genus Fusicatenibacter could be useful in the treatment or prevention of autism spectrum disorders, and in particular treating or preventing the gastrointestinal dysfunction which is associated with autism spectrum disorders.

Sequences

SEQ ID NO: 1-Fusicatenibacter saccharivorans gene for 16S ribosomal RNA,
partial sequence, strain: HT03-11 (AB698910)
tggctcagga tgaacgctgg cggcgtgctt aacacatgca agtcgagcga agcagttaag
aagattyttc ggatgattct tgactgactg agcggcggac gggtgagtaa cgcgtgggtg
acctgcccca taccggggga taacagctgg aaacggctgc taataccgca taagcgcaca
gagctgcatg gctcggtgtg aaaaactccg gtggtatggg atgggcccgc gtctgattag
gcagttggcg gggtaacggc ccaccaaacc gacgatcagt agccggcctg agagggcgac
cggccacatt gggactgaga cacggcccaa actcctacgg gaggcagcag tggggaatat
tgcacaatgg gggaaaccct gatgcagcga cgccgcgtga gcgaagaagt atttcggtat
gtaaagctct atcagcaggg aagataatga cggtacctga ctaagaagcc ccggctaact
acgtgccagc agccgcggta atacgtaggg ggcaagcgtt atccggattt actgggtgta
aagggagcgt agacggcaag gcaagtctga tgtgaaaacc cagggcttaa ccctgggact
gcattggaaa ctgtctggct cgagtgccgg agaggtaagc ggaattccta gtgtagcggt
gaaatgcgta gatattagga agaacaccag tggcgaaggc ggcttactgg acggtaactg
acgttgaggc tcgaaagcgt ggggagcaaa caggattaga taccctggta gtccacgccg
taaacgatga atgctaggtg ttggggagca aagctcttcg gtgccgccgc aaacgcatta
agcattccac ctggggagta cgttcgcaag aatgaaactc aaaggaattg acggggaccc
gcacaagcgg tggagcatgt ggtttaattc gaagcaacgc gaagaacctt accaggtctt
gacatcccga tgaccggccc gtaacggggc cttctcttcg gagcattgga gacaggtggt
gcatggttgt cgtcagctcg tgtcgtgaga tgttgggtta agtcccgcaa cgagcgcaac
ccttatcctc agtagccagc aggtaaagct gggcactctg tggagactgc cagggataac
ctggaggaag gtggggatga cgtcaaatca tcatgcccct tatgatctgg gctacacacg
tgctacaatg gcgtaaacaa agggaggcaa agccgcgagg tggagcaaat cccaaaaata
acgtctcagt tcggactgca gtctgcaact cgactgcacg aagctggaat cgctagtaat
cgcgaatcag aatgtcgcgg tgaatacgtt cccgggtctt gtacacaccg cccgtcacac
catgggagtt ggtaacgccc gaagtcagtg acccaacctt tta
SEQ ID NO: 2-Fusicatenibacter saccharivorans gene for 16S ribosomal RNA,
partial sequence, strain: KO-38
ttgactgagc ggcggacggg tgagtaacgc gtgggtgacc tgccccatac cgggggataa
cagctggaaa cggctgctaa taccgcataa gcgcacagag ctgcatggct cggtgtgaaa
aactccggtg gtatgggatg ggcccgcgtc tgattaggca gttggcgggg taacggccca
ccaaaccgac gatcagtagc cggcctgaga gggcgaccgg ccacattggg actgagacac
ggcccaaact cctacgggag gcagcagtgg ggaatattgc acaatggggg aaaccctgat
gcagcgacgc cgcgtgagcg aagaagtatt tcggtatgta aagctctatc agcagggaag
ataatgacgg tacctgacta agaagccccg gctaactacg tgccagcagc cgcggtaata
cgtagggggc aagcgttatc cggatttact gggtgtaaag ggagcgtaga cggcaaggca
agtctgatgt gaaaacccag ggcttaaccc tgggactgca ttggaaactg tctggctcga
gtgccggaga ggtaagcgga attcctagtg tagcggtgaa atgcgtagat attaggaaga
acaccagtgg cgaaggcggc ttactggacg gtaactgacg ttgaggctcg aaagcgtggg
gagcaaacag gattagatac cctggtagtc cacgccgtaa acgatgaatg ctaggtgttg
gggagcaaag ctcttcggtg ccgccgcaaa cgcattaagc attccacctg gggagtacgt
tcgcaagaat gaaactcaaa ggaattgacg gggacccgca caagcggtgg agcatgtggt
ttaattcgaa gcaacgcgaa gaaccttacc aggtcttgac atcccgatga ccggcccgta
acggggcctt ctcttcggag cattggagac aggtggtgca tggttgtcgt cagctcgtgt
cgtgagatgt tgggttaagt cccgcaacga gcgcaaccct tatcctcagt agccagcagg
taaagctggg cactctgtgg agactgccag ggataacctg gaggaaggtg gggatgacgt
caaatcatca tgccccttat gatctgggct acacacgtgc tacaatggcg taaacaaagg
gaggcaaagc cgcgaggtgg agcaaatccc aaaaataacg tctcagttcg gactgcagtc
tgcaactcga ctgcacgaag ctggaatcgc tagtaatcgc gaatcagaat gtcgcggtga
atacgttccc gggtcttgta cacaccgccc gtcacaccat gggagttggt aacgcccgaa
gtcagtgacc caacctttta ggagggagct gccgaaggcg ggactgataa ctggggtgaa
gtcgtaacaa gg
SEQ ID NO: 3-Fusicatenibacter saccharivorans gene for 16S ribosomal RNA,
partial sequence, strain: TT-111
gctcaggatg aacgctggcg gcgtgcttaa cacatgcaag tcgagcgaag cagttaagaa
gattyttcgg atgattttta actgactgag cggcggacgg gtgagtaacg cgtgggtgac
ctgccccata ccgggggata acagctggaa acggctgcta ataccgcata agcgcacaga
gctgcatggc tcggtgtgaa aaactccggt ggtatgggat gggcccgcgt ctgattaggc
agttggcggg gtaacggccc accaaaccga cgatcagtag ccggcctgag agggcgaccg
gccacattgg gactgagaca cggcccaaac tcctacggga ggcagcagtg gggaatattg
cacaatgggg gaaaccctga tgcagcgacg ccgcgtgagc gaagaagtat ttcggtatgt
aaagctctat cagcagggaa gataatgacg gtacctgact aagaagcccc ggctaactac
gtgccagcag ccgcggtaat acgtaggggg caagcgttat ccggatttac tgggtgtaaa
gggagcgtag acggcaaggc aagtctgatg tgaaaaccca gggcttaacc ctgggactgc
attggaaact gtctggctcg agtgccggag aggtaagcgg aattcctagt gtagcggtga
aatgcgtaga tattaggaag aacaccagtg gcgaaggcgg cttactggac ggtaactgac
gttgaggctc gaaagcgtgg ggagcaaaca ggattagata ccctggtagt ccacgccgta
aacgatgaat gctaggtgtt ggggagcaaa gctcttcggt gccgccgcaa acgcattaag
cattccacct ggggagtacg ttcgcaagaa tgaaactcaa aggaattgac ggggacccgc
acaagcggtg gagcatgtgg tttaattcga agcaacgcga agaaccttac caggtcttga
catcccgatg accggcccgt aacggggcct tctcttcgga gcattggaga caggtggtgc
atggttgtcg tcagctcgtg tcgtgagatg ttgggttaag tcccgcaacg agcgcaaccc
ttatcctcag tagccagcag gtaaagctgg gcactctgtg gagactgcca gggataacct
ggaggaaggt ggggatgacg tcaaatcatc atgcccctta tgatctgggc tacacacgtg
ctacaatggc gtaaacaaag ggaggcaaag ccgcgaggtg gagcaaatcc caaaaataac
gtctcagttc ggactgcagt ctgcaactcg actgcacgaa gctggaatcg ctagtaatcg
cgaatcagaa tgtcgcggtg aatacgttcc cgggtcttgt acacaccgcc cgtcacacca
tgggagttgg taacgcccga agtcagtgac ccaaccgttt acggagggag ctgccgaagg
cgggactgat aactggggtg aagtcgtaac aaggtagccg tateggaagg tgcggctgga
tcacctcc
SEQ ID NO: 4-16S ribosomal RNA of strain deposited under accession number
NCIMB 43527
ggcccttaacccgaaattcggcagctccctcctgaaaggttgggtcactgacttcgggcgttaccaa
ctcccatggtgtgacgggcggtgtgtacaagacccgggaacgtattcaccgcgacattctgattcgc
gattactagcgattccagcttcgtgcagtcgagttgcagactgcagtccgaactgagacgttatttt
tgggatttgctccacctcgcggctttgcctccctttgtttacgccattgtagcacgtgtgtagccca
gatcataaggggcatgatgatttgacgtcatccccaccttcctccaggttatccctggcagtctcca
cagagtgcccagctttacctgctggctactgaggataagggttgcgctcgttgcgggacttaaccca
acatctcacgacacgagctgacgacaaccatgcaccacctgtctccaatgctccgaagagaaggccc
cgttacgggccggtcatcgggatgtcaagacctggtaaggttcttcgcgttgcttcgaattaaacca
catgctccaccgcttgtgcgggtccccgtcaattcctttgagtttcattcttgcgaacgtactcccc
aggtggaatgcttaatgcgtttgcggcggcaccgaagagctttgctccccaacacctagcattcatc
gtttacggcgtggactaccagggtatctaatcctgtttgctccccacgctttcgagcctcaacgtca
gttaccgtccagtaagccgccttcgccactggtgttcttcctaatatctacgcatttcaccgctaca
ctaggaattccgcttacctctccggcactcgagccagacagtttccaatgcagtcccagggttaagc
cctgggttttcacatcagacttgccttgccgtctacgctccctttacacccagtaaatccggataac
gcttgccccctacgtattaccgcggctgctggcacgtagttagccggggcttcttagtcaggtaccg
tcattatcttccctgctgatagagctttacataccgaaatacttcttcgctcacgcggcgtcgctgc
atcagggtttcccccattgtgcaatattccccactgctgcctcccgtaggaatttgggccgtgtctc
agtcccaatgtggccggtcgccctctcaggccggctactgatcgtcggtttggtgggccgttacccc
gccaactgcctaatcagacgcgggcccatcccataccaccggaattttttcaaccgaagccaggcaa
ctttgtggcccttatgcgggtattaagaaccccttttcccgctttttttcccccggttatgggggag
ggcccccccccgtttttcccccccccccccccctcaataatagtaaaaaataccccaaaaaaaacca
aaacgccccccacgcggggggg

### EMBODIMENTS

1. A composition comprising a live bacterial strain of the genus Fusicatenibacter for use in therapy.
2. A composition comprising a bacterial strain of the genus Fusicatenibacter for use in a method of treating or preventing a disease or disorder selected from the group consisting of: a central nervous system disease, disorder or condition, impaired gut barrier function, a disease which is associated with increased intestinal permeability, an inflammatory disease, a metabolic disease and a cardiovascular disease.
3. A composition comprising a bacterial strain that has a 16s rRNA sequence that has at least 95% sequence similarity to SEQ ID NOs:1, 2, 3 or 4 for use in a method of treating or preventing a disease or disorder selected from the group consisting of: a central nervous system disease, disorder or condition, impaired gut barrier function, a disease which is characterised by increased intestinal permeability, an inflammatory disease, a metabolic or a cardiovascular disease.
4. A composition according to embodiment 3, wherein the bacterial strain has a 16s rRNA sequence that is at least 96%, 97%, 98%, 99%, 99.5%, 99.6%, 99.7%, 99.8% or 99.9% similar to SEQ ID NOs: 1, 2, 3 or 4.
5. The composition according to any one of embodiments 2 to 4, wherein the bacterial strain is live.
6. The composition for use according to any of the preceding embodiments, wherein the composition is for use in the treatment of patient identified as having impaired gut barrier function.
7. The composition for use according to any preceding embodiment, wherein the central nervous system disease or disorder is mediated by the microbiota-gut-brain axis.
8. The composition for use according to any preceding embodiment, wherein the composition is for use in a method of modulating the microbiota-gut-brain axis.
9. The composition for use according to any preceding embodiment, wherein the composition is for use in a method of treating or preventing a neurodegenerative disease, a neurodevelopmental disorder or a neuropsychiatric condition.
10. A composition for use according to embodiment 9, wherein the neurodevelopmental disorder or neuropsychiatric condition is selected from the group consisting of autism spectrum disorders (ASDs); child developmental disorder; obsessive compulsive disorder (OCD); major depressive disorder; depression; seasonal affective disorder; anxiety disorders; chronic fatigue syndrome (myalgic encephalomyelitis); stress disorder; post-traumatic stress disorder; schizophrenia spectrum disorders; schizophrenia; bipolar disorder; psychosis; mood disorder; chronic pain; Guillain-Barré syndrome and meningitis, dementia, including Lewy body, vascular and frontotemporal dementia; primary progressive aphasia; mild cognitive impairment; HIV-related cognitive impairment, and corticobasal degeneration.
11. A composition comprising bacterial strain of the genus Fusicatenibacter for use in a method of treating or preventing autism spectrum disorder.
12. A composition for use according to embodiment 11 wherein the composition is for use in a method of reducing or preventing autism.
13. A composition for use according to embodiment 12, wherein the composition prevents, reduces or alleviates stereotyped, repetitive, compulsive and/or anxious behaviour.
14. The composition for use according to embodiments 1-9, wherein the composition is for use in a method of treating or preventing a neurodegenerative disease.
15. The composition for use according to embodiment 14, wherein the neurodegenerative disease is be selected from the group consisting of Parkinson's disease, including progressive supranuclear palsy, progressive supranuclear palsy, Steele-Richardson-Olszewski syndrome, normal pressure hydrocephalus, vascular or arteriosclerotic parkinsonism and drug-induced parkinsonism; Alzheimer's disease, including Benson's syndrome; multiple sclerosis; Huntington's disease; amyotrophic lateral sclerosis; Lou Gehrig's disease; motor inflammatory neurone disease; prion disease; spinocerebellar ataxia or spinal muscular atrophy.
16. The composition for use according to embodiment 1-9, wherein the composition is for use in a method of treating or preventing an inflammatory disease.
17. The composition for use according to embodiment 16, wherein the inflammatory disease is inflammatory bowel disease, sepsis and acute lung injury
18. The composition for use according to embodiment 1-9, wherein the composition is for use in a method of treating or preventing a metabolic disease.
19. The composition for use according to embodiment 1-9, wherein the composition is for use in a method of treating or preventing a cardiovascular disease.
20. The composition for use according to embodiment 19, wherein the cardiovascular disease is atherosclerosis, hypertension, coronary artery disease, angina and myocardial infarction, heart arrhythmia, heart failure, valvular heart disease, Pericardial disease, stroke, hypertensive heart disease, rheumatic heart disease, cardiomyopathy, congenital heart disease, carditis, aortic aneurysms, peripheral artery disease, thromboembolic disease, or venous thrombosis.
21. The composition for use according to embodiment 1-9, wherein the composition is for use in a method of treating or preventing a disease which is associated with increased intestinal permeability.
22. The composition for use according to embodiment 21, wherein the disease which is associated with increased intestinal permeability is gastric ulcers, infectious diarrhea, irritable bowel syndrome, functional GI diseases, inflammatory bowel disease, celiac disease and cancer (esophagus, colorectal), food allergies, acute inflammation (sepsis, systemic inflammatory response syndrome (SIRS), multiple organ failure), obesity, metabolic diseases (e.g. NAFLD and NASH, type 2 diabetes mellitus or cardiovascular diseases) and chronic inflammation (e.g. arthritis).
23. The composition for use according to any preceding embodiment, wherein the composition is not for use in treating or preventing a Clostridium difficile infection
24. The composition for use according to embodiments 1-23, wherein the bacterial strain has a 16s rRNA sequence that is at least 95%, 96%, 97%, 98%, 99%, 99.5%, 99.6%, 99.7%, 99.8% or 99.9% identical to the 16s rRNA sequence of a bacterial species Fusicatenibacter saccharivorans.
25. The composition for use according to any of embodiments 1-23, wherein the bacterial strain has a 16s rRNA sequence that is at least 95%, 96%, 97%, 98%, 99%, 99.5%, 99.6%, 99.7%, 99.8% or 99.9% identical to SEQ ID Nos: 1, 2, 3 or 4.
26. The composition for use according to embodiments 1-25, wherein the bacterial strain is the species Fusicatenibacter saccharivorans.
27. The composition of any preceding embodiment, wherein the composition is for oral administration.
28. The composition for use according to any preceding embodiment, wherein the composition comprises one or more pharmaceutically acceptable excipients or carriers.
29. The composition for use according to any preceding embodiment, wherein the bacterial strain is viable.
30. The composition for use according to any preceding embodiment, wherein the bacterial strain is lyophilised.
31. The composition for use according to any preceding embodiment, wherein the bacterial strain is capable of partially or totally colonising the intestine.
32. The composition for use according to any preceding embodiment, wherein the composition comprises a single species of Fusicatenibacter.
33. The composition for use according to any preceding embodiment, wherein the composition comprises a single strain of Fusicatenibacter.
34. The composition for use according to embodiment 1-32, which comprises the Fusicatenibacter bacterial strain as part of a microbial consortium.
35. A food product comprising the composition of any preceding embodiment, for the use of any preceding embodiment.
36. A vaccine composition comprising the composition of any preceding embodiment, for the use of any preceding embodiment.
37. A method of treating or preventing a disease comprising administering a composition comprising a live bacterial strain of the genus Fusicatenibacter to a patient in need thereof.
38. Use of a live bacterial strain of the genus Fusicatenibacter in the manufacture of a medicament for use in prevention or treatment of a disease.
39. A method of treating or preventing a central nervous system disease, a disease which is associated with increased intestinal permeability, an inflammatory disease, a metabolic disease or a cardiovascular disease comprising administering a composition comprising a live bacterial strain of the genus Fusicatenibacter to a patient in need thereof.
40. Use of a bacterial strain of the genus Fusicatenibacter in the manufacture of a medicament for use in prevention or treatment of a central nervous system disease, a disease which is associated with increased intestinal permeability, an inflammatory disease, a metabolic disease or a cardiovascular disease.
41. The use of embodiment 40, wherein the bacterial strain is live.
42. A method of treating or preventing autism comprising administering a composition comprising a bacterial strain of the genus Fusicatenibacter to a patient in need thereof.
43. Use of a bacterial strain of the genus Fusicatenibacter in the manufacture of a medicament for use in prevention or treatment autism.
44. A composition comprising the cell of any one of embodiment 43 for the use of any of embodiments 1-34.
45. The composition of embodiment 44, comprising a pharmaceutically acceptable carrier or excipient.
46. A cell of the Fusicatenibacter saccharivorans strain deposited under accession number NCIMB 43527, or a derivative thereof.
47. A composition comprising the cell of embodiment 46.
48. The composition of embodiment 47, comprising a pharmaceutically acceptable carrier or excipient.
49. The cell of embodiment 46 or the composition of embodiment 47 or 48, for the use of any of embodiments 1-34.

### REFERENCES

[1] Spor et al. (2011) Nat Rev Microbiol. 9(4):279-90.
[2] Eckburg et al. (2005) Science. 10; 308(5728):1635-8.
[3] Tap et al. (2009), Environ Microbiol, 11(10):2574-84.
[4] Macpherson et al. (2001) Microbes Infect. 3(12):1021-35
[5] Macpherson et al. (2002) Cell Mol Life Sci. 59(12):2088-96.
[6] Mazmanian et al. (2005) Cell 15; 122(1):107-18.
[7] Frank et al. (2007) PNAS 104(34):13780-5.
[8] Scanlan et al. (2006) J Cln Microbiol. 44(11):3980-8.
[9] Kang et al. (2010) Inflamm Bowel Dis. 16(12):2034-42.
[10] Machiels et al. (2013) Gut. 63(8):1275-83.
[11] Lopetuso et al. (2013), Gut Pathogens, 5: 23
[12] WO 2013/050792
[13] WO 03/046580
[14] WO 2013/008039
[15] WO 2014/167338
[16] Lee and Lee (2014) World J Gastroenterol. 20(27): 8886-8897.
[17] Takeshita et al. (2016) Inflamm Bowel Dis. 22, 12, 2802-2810
[18] Takada et al (2013) Int J Syst Evol Microbiol 63, 3691-3696
[19] Chun et al (2007). Int J Syst Evol Microbiol 57, 2259-2261.
[20] Masco et al. (2003) Systematic and Applied Microbiology, 26:557-563.
[21] Srntkova et al. (2011) J. Microbiol. Methods, 87(1):10-6.
[22] Boyd et al. (2010) Journal of Early Intervention 32, 2, 75-98
[23] Wang et al. (2016) J Neurogastroenterol Motil 22: 589-605.
[24] Lee et al (2008) European J. Cell Biology 87:389-397
[25] Routy et al (2016) Int J Tryptophan Res. 9:67-77.
[26] Kratsman et al (2016) Neuropharmacology, 102, 136-145
[27] Bischoff et al. (2014) BMC Gastroenterology 14:189
[28] Vinolo et al. (2011) Nutrients. 3(10): 858-876.
[29] Chambers et al. (2018) Curr Nutr Rep. 7(4): 198-206.
[30] Ahmadmehrabil & Tang, (2017) Curr Opin Cardiol.; 32(6): 761-766.
[31] Mitropoulou et al. (2013) J Nutr Metab. (2013) 716861.
[32] Kailasapathy et al. (2002) Curr Issues Intest Microbiol. 3(2):39-48.
[33] Miyamoto-Shinohara et al. (2008) J. Gen. Appl. Microbiol., 54, 9-24.
[34] Cryopreservation and Freeze-Drying Protocols, ed. by Day and McLellan, Humana Press.
[35] Leslie et al. (1995) Appl. Environ. Microbiol. 61, 3592-3597.
[36] Handbook of Pharmaceutical Excipients, 2nd Edition, (1994), Edited by A Wade and PJ Weller
[37] Remington's Pharmaceutical Sciences, Mack Publishing Co. (A. R. Gennaro edit. 1985)
[38] US 2016/0067188
[39] Handbook of Microbiological Media, Fourth Edition (2010) Ronald Atlas, CRC Press.
[40] Maintaining Cultures for Biotechnology and Industry (1996) Jennie C. Hunter-Cevera, Academic Press
[41] Strobel (2009) Methods Mol Biol. 581:247-61.
[42] Gennaro (2000) Remington: The Science and Practice of Pharmacy. 20th edition, ISBN: 0683306472.
[43] Molecular Biology Techniques: An Intensive Laboratory Course, (Ream et al., eds., 1998, Academic Press).
[44] Methods In Enzymology (S. Colowick and N. Kaplan, eds., Academic Press, Inc.)
[45] Handbook of Experimental Immunology, Vols. I-IV (D. M. Weir and C. C. Blackwell, eds, 1986, Blackwell Scientific Publications)
[46] Sambrook et al. (2001) Molecular Cloning: A Laboratory Manual, 3rd edition (Cold Spring Harbor Laboratory Press).
[47] Handbook of Surface and Colloidal Chemistry (Birdi, K. S. ed., CRC Press, 1997)
[48] Ausubel et al. (eds) (2002) Short protocols in molecular biology, 5th edition (Current Protocols).
[49] PCR (Introduction to Biotechniques Series), 2nd ed. (Newton & Graham eds., 1997, Springer Verlag)
[50] Current Protocols in Molecular Biology (F. M. Ausubel et al., eds., 1987) Supplement 30
[51] Smith & Waterman (1981) Adv. Appl. Math. 2: 482-489.
[52] Golubeva et al (2017) EBioMedicine, 24, 166-178
[53] Meyza and Blanchard (2017) Neurosci Biobehav Rev 76(Pt A): 99-110.
[54] Crawley (2012) Dialogues in Clinical Neuroscience—Vol 14, No. 3

1.-17. (canceled)

18. A composition comprising a bacterial strain of the genus Fusicatenibacter, wherein the Fusicatenibacter bacterial strain comprises a 16s rRNA gene sequence with at least 95% sequence identity to the 16s rRNA gene sequence of SEQ ID NOs: 1, 2, 3, or 4, and wherein the Fusicatenibacter bacterial strain is lyophilized.

19. The composition of claim 18, wherein the Fusicatenibacter bacterial strain is viable.

20. The composition of claim 18, wherein the composition is encapsulated.

21. The composition of claim 18, wherein the Fusicatenibacter bacterial strain is present in an amount that comprises from about 1×10³ to about 1×10¹¹ colony forming units per gram (CFU/g) of the Fusicatenibacter bacterial strain with respect to a total weight of the composition.

22. The composition of claim 18, wherein the Fusicatenibacter bacterial strain is present in an amount that comprises from about 1×10⁸ to about 1×10¹⁰ CFU/g of the Fusicatenibacter bacterial strain with respect to a total weight of the composition.

23. The composition of claim 18, wherein the sequence identity is determined by a Smith-Waterman homology search algorithm using an affine gap search with a gap open penalty of 12 and a gap extension penalty of 2.

24. The composition of claim 18, wherein the Fusicatenibacter bacterial strain comprises a 16s rRNA gene sequence with at least 98% sequence identity to the 16s rRNA gene sequence of SEQ ID NOs: 1, 2, 3, or 4, determined by a Smith-Waterman homology search algorithm using an affine gap search with a gap open penalty of 12 and a gap extension penalty of 2.

25. The composition of claim 18, wherein the Fusicatenibacter bacterial strain comprises a 16s rRNA gene sequence with at least 99% sequence identity to the 16s rRNA gene sequence of SEQ ID NOs: 1, 2, 3, or 4, determined by a Smith-Waterman homology search algorithm using an affine gap search with a gap open penalty of 12 and a gap extension penalty of 2.

26. The composition of claim 18, wherein the Fusicatenibacter bacterial strain is the bacterial strain deposited under accession number NCIMB 43527.

27. The composition of claim 18, wherein the Fusicatenibacter bacterial strain is live.

28. The composition of claim 18, wherein the Fusicatenibacter bacterial strain colonizes an intestine when administered to a subject.

29. The composition of claim 18, formulated for oral delivery.

30. The composition of claim 18, formulated for rectal delivery.

31. The composition of claim 18, wherein the composition is formulated as a tablet, a capsule, or powder.

32. The composition of claim 18, wherein the composition does not comprise a therapeutically effective amount of additional bacteria strains.

33. The composition of claim 18, further comprising a pharmaceutically acceptable excipient, diluent, or carrier.

34. The composition of claim 33, wherein the composition comprises a pharmaceutically acceptable carrier, wherein the pharmaceutically acceptable carrier is selected from the group consisting of lactose, starch, glucose, methyl cellulose, magnesium stearate, mannitol, and sorbitol.

35. The composition of claim 18, further comprising a preservative, an antioxidant, or a stabilizer.

36. A method of treating a disease or condition associated with impaired gut barrier function in a subject in need thereof, the method comprising: administering to the subject a composition comprising a bacterial strain of the genus Fusicatenibacter, wherein the Fusicatenibacter bacterial strain comprises a 16s rRNA gene sequence with at least 95% sequence identity to the 16s rRNA gene sequence of SEQ ID NOs: 1, 2, 3, or 4, wherein subject has an increased intestinal permeability, and wherein the administering is effective to reduce the level of intestinal permeability compared to the level of intestinal permeability in the subject prior to the administering.

37. The method of claim 36, wherein the disease or condition associated with impaired gut barrier function comprises gastric ulcers, infectious diarrhea, irritable bowel syndrome, functional gastrointestinal (GI) diseases, inflammatory bowel disease, celiac disease, celiac cancer, esophagus cancer, colorectal cancer, food allergies, acute inflammation, sepsis, systemic inflammatory response syndrome (SIRS), multiple organ failure, obesity, metabolic diseases, non-alcoholic fatty liver disease (NAFLD), nonalcoholic steatohepatitis (NASH), type 2 diabetes mellitus, cardiovascular disease, chronic inflammation, or arthritis.


SEQUENCE LISTING (4 sequences)

SEQ ID NO: 1 - DNA, 1423 bp, Fusicatenibacter saccharivorans
  Fusicatenibacter saccharivorans strain HT03-11
tggctcaggatgaacgctggcggcgtgcttaacacatgcaagtcgagcgaagcagttaagaagattyttcggatgattcttgactgactgagcggcggacgggtgagtaacgcgtgggtgacctgccccataccgggggataacagctggaaacggctgctaataccgcataagcgcacagagctgcatggctcggtgtgaaaaactccggtggtatgggatgggcccgcgtctgattaggcagttggcggggtaacggcccaccaaaccgacgatcagtagccggcctgagagggcgaccggccacattgggactgagacacggcccaaactcctacgggaggcagcagtggggaatattgcacaatgggggaaaccctgatgcagcgacgccgcgtgagcgaagaagtatttcggtatgtaaagctctatcagcagggaagataatgacggtacctgactaagaagccccggctaactacgtgccagcagccgcggtaatacgtagggggcaagcgttatccggatttactgggtgtaaagggagcgtagacggcaaggcaagtctgatgtgaaaacccagggcttaaccctgggactgcattggaaactgtctggctcgagtgccggagaggtaagcggaattcctagtgtagcggtgaaatgcgtagatattaggaagaacaccagtggcgaaggcggcttactggacggtaactgacgttgaggctcgaaagcgtggggagcaaacaggattagataccctggtagtccacgccgtaaacgatgaatgctaggtgttggggagcaaagctcttcggtgccgccgcaaacgcattaagcattccacctggggagtacgttcgcaagaatgaaactcaaaggaattgacggggacccgcacaagcggtggagcatgtggtttaattcgaagcaacgcgaagaaccttaccaggtcttgacatcccgatgaccggcccgtaacggggccttctcttcggagcattggagacaggtggtgcatggttgtcgtcagctcgtgtcgtgagatgttgggttaagtcccgcaacgagcgcaacccttatcctcagtagccagcaggtaaagctgggcactctgtggagactgccagggataacctggaggaaggtggggatgacgtcaaatcatcatgccccttatgatctgggctacacacgtgctacaatggcgtaaacaaagggaggcaaagccgcgaggtggagcaaatcccaaaaataacgtctcagttcggactgcagtctgcaactcgactgcacgaagctggaatcgctagtaatcgcgaatcagaatgtcgcggtgaatacgttcccgggtcttgtacacaccgcccgtcacaccatgggagttggtaacgcccgaagtcagtgacccaacctttta

SEQ ID NO: 2 - DNA, 1392 bp, Fusicatenibacter saccharivorans
  Fusicatenibacter saccharivorans strain KO-38
ttgactgagcggcggacgggtgagtaacgcgtgggtgacctgccccataccgggggataacagctggaaacggctgctaataccgcataagcgcacagagctgcatggctcggtgtgaaaaactccggtggtatgggatgggcccgcgtctgattaggcagttggcggggtaacggcccaccaaaccgacgatcagtagccggcctgagagggcgaccggccacattgggactgagacacggcccaaactcctacgggaggcagcagtggggaatattgcacaatgggggaaaccctgatgcagcgacgccgcgtgagcgaagaagtatttcggtatgtaaagctctatcagcagggaagataatgacggtacctgactaagaagccccggctaactacgtgccagcagccgcggtaatacgtagggggcaagcgttatccggatttactgggtgtaaagggagcgtagacggcaaggcaagtctgatgtgaaaacccagggcttaaccctgggactgcattggaaactgtctggctcgagtgccggagaggtaagcggaattcctagtgtagcggtgaaatgcgtagatattaggaagaacaccagtggcgaaggcggcttactggacggtaactgacgttgaggctcgaaagcgtggggagcaaacaggattagataccctggtagtccacgccgtaaacgatgaatgctaggtgttggggagcaaagctcttcggtgccgccgcaaacgcattaagcattccacctggggagtacgttcgcaagaatgaaactcaaaggaattgacggggacccgcacaagcggtggagcatgtggtttaattcgaagcaacgcgaagaaccttaccaggtcttgacatcccgatgaccggcccgtaacggggccttctcttcggagcattggagacaggtggtgcatggttgtcgtcagctcgtgtcgtgagatgttgggttaagtcccgcaacgagcgcaacccttatcctcagtagccagcaggtaaagctgggcactctgtggagactgccagggataacctggaggaaggtggggatgacgtcaaatcatcatgccccttatgatctgggctacacacgtgctacaatggcgtaaacaaagggaggcaaagccgcgaggtggagcaaatcccaaaaataacgtctcagttcggactgcagtctgcaactcgactgcacgaagctggaatcgctagtaatcgcgaatcagaatgtcgcggtgaatacgttcccgggtcttgtacacaccgcccgtcacaccatgggagttggtaacgcccgaagtcagtgacccaaccttttaggagggagctgccgaaggcgggactgataactggggtgaagtcgtaacaagg

SEQ ID NO: 3 - DNA, 1508 bp, Fusicatenibacter saccharivorans
  Fusicatenibacter saccharivorans strain TT-111
gctcaggatgaacgctggcggcgtgcttaacacatgcaagtcgagcgaagcagttaagaagattyttcggatgatttttaactgactgagcggcggacgggtgagtaacgcgtgggtgacctgccccataccgggggataacagctggaaacggctgctaataccgcataagcgcacagagctgcatggctcggtgtgaaaaactccggtggtatgggatgggcccgcgtctgattaggcagttggcggggtaacggcccaccaaaccgacgatcagtagccggcctgagagggcgaccggccacattgggactgagacacggcccaaactcctacgggaggcagcagtggggaatattgcacaatgggggaaaccctgatgcagcgacgccgcgtgagcgaagaagtatttcggtatgtaaagctctatcagcagggaagataatgacggtacctgactaagaagccccggctaactacgtgccagcagccgcggtaatacgtagggggcaagcgttatccggatttactgggtgtaaagggagcgtagacggcaaggcaagtctgatgtgaaaacccagggcttaaccctgggactgcattggaaactgtctggctcgagtgccggagaggtaagcggaattcctagtgtagcggtgaaatgcgtagatattaggaagaacaccagtggcgaaggcggcttactggacggtaactgacgttgaggctcgaaagcgtggggagcaaacaggattagataccctggtagtccacgccgtaaacgatgaatgctaggtgttggggagcaaagctcttcggtgccgccgcaaacgcattaagcattccacctggggagtacgttcgcaagaatgaaactcaaaggaattgacggggacccgcacaagcggtggagcatgtggtttaattcgaagcaacgcgaagaaccttaccaggtcttgacatcccgatgaccggcccgtaacggggccttctcttcggagcattggagacaggtggtgcatggttgtcgtcagctcgtgtcgtgagatgttgggttaagtcccgcaacgagcgcaacccttatcctcagtagccagcaggtaaagctgggcactctgtggagactgccagggataacctggaggaaggtggggatgacgtcaaatcatcatgccccttatgatctgggctacacacgtgctacaatggcgtaaacaaagggaggcaaagccgcgaggtggagcaaatcccaaaaataacgtctcagttcggactgcagtctgcaactcgactgcacgaagctggaatcgctagtaatcgcgaatcagaatgtcgcggtgaatacgttcccgggtcttgtacacaccgcccgtcacaccatgggagttggtaacgcccgaagtcagtgacccaaccgtttacggagggagctgccgaaggcgggactgataactggggtgaagtcgtaacaaggtagccgtatcggaaggtgcggctggatcacctcc

SEQ ID NO: 4 - DNA, 1429 bp, Fusicatenibacter saccharivorans
ggcccttaacccgaaattcggcagctccctcctgaaaggttgggtcactgacttcgggcgttaccaactcccatggtgtgacgggcggtgtgtacaagacccgggaacgtattcaccgcgacattctgattcgcgattactagcgattccagcttcgtgcagtcgagttgcagactgcagtccgaactgagacgttatttttgggatttgctccacctcgcggctttgcctccctttgtttacgccattgtagcacgtgtgtagcccagatcataaggggcatgatgatttgacgtcatccccaccttcctccaggttatccctggcagtctccacagagtgcccagctttacctgctggctactgaggataagggttgcgctcgttgcgggacttaacccaacatctcacgacacgagctgacgacaaccatgcaccacctgtctccaatgctccgaagagaaggccccgttacgggccggtcatcgggatgtcaagacctggtaaggttcttcgcgttgcttcgaattaaaccacatgctccaccgcttgtgcgggtccccgtcaattcctttgagtttcattcttgcgaacgtactccccaggtggaatgcttaatgcgtttgcggcggcaccgaagagctttgctccccaacacctagcattcatcgtttacggcgtggactaccagggtatctaatcctgtttgctccccacgctttcgagcctcaacgtcagttaccgtccagtaagccgccttcgccactggtgttcttcctaatatctacgcatttcaccgctacactaggaattccgcttacctctccggcactcgagccagacagtttccaatgcagtcccagggttaagccctgggttttcacatcagacttgccttgccgtctacgctccctttacacccagtaaatccggataacgcttgccccctacgtattaccgcggctgctggcacgtagttagccggggcttcttagtcaggtaccgtcattatcttccctgctgatagagctttacataccgaaatacttcttcgctcacgcggcgtcgctgcatcagggtttcccccattgtgcaatattccccactgctgcctcccgtaggaatttgggccgtgtctcagtcccaatgtggccggtcgccctctcaggccggctactgatcgtcggtttggtgggccgttaccccgccaactgcctaatcagacgcgggcccatcccataccaccggaattttttcaaccgaagccaggcaactttgtggcccttatgcgggtattaagaaccccttttcccgctttttttcccccggttatgggggagggcccccccccgtttttcccccccccccccccctcaataatagtaaaaaataccccaaaaaaaaccaaaacgccccccacgcggggggg